(19) United States
(12) Patent Application Publication
(10) Pub. No.: US 2012/0142768 A1
(43) Pub. Date: Jun. 7, 2012

(54) FORMULATIONS INCLUDING AMIODARONE AND SALTS THEREOF AND METHODS OF THEIR MANUFACTURE AND USE

(75) Inventors: Gerold L. Mosher, Kansas City, MO (US); Stephen Machatha, Burlington, MA (US); Daniel Cushing, Phoenixville, PA (US); Bushra Agha, Chapel Hill, NC (US)

(73) Assignees: BAXTER HEALTHCARE S.A., Glattpark (Opfikon) (CH); BAXTER INTERNATIONAL INC., Deerfield, IL (US)

(21) Appl. No.: 13/156,022

(22) Filed: Jun. 8, 2011

Related U.S. Application Data
(60) Provisional application No. 61/353,927, filed on Jun. 11, 2010.

(51) Int. Cl. A61K 31/343; A61J 1/10; A61P 9/06

(52) U.S. Cl. 514/469; 604/408

(57) ABSTRACT

The invention encompasses ready to administer liquid formulations including amiodarone and a substituted cyclodextrin. The invention also encompasses methods of making the liquid formulations to provide acceptable concentrations of amiodarone suitable for parenteral administration. The liquid formulations of the invention are formulations included, for example, in a ready to use intravenous bag, bottle or syringe.

### PRIORITY CLAIM

This application claims the benefit of U.S. Provisional Patent Application Ser. No. 61/353,927 filed on Jun. 11, 2010, the entire disclosure of which is hereby incorporated by reference.

### I. FIELD OF THE INVENTION

The invention encompasses ready to use liquid formulations including amiodarone or a salt thereof and a substituted cyclodextrin. The invention also encompasses methods of making the liquid formulations to provide acceptable concentrations of amiodarone suitable for parenteral administration while avoiding the formation of gel or particulates. The liquid formulations of the invention are formulations, for example, included in an intravenous bag or bottle.

### II. BACKGROUND OF THE INVENTION

Amiodarone is approved for the treatment of life-threatening ventricular tachyarrhythmias. Amiodarone is also useful in treating less severe ventricular arrhythmias and many supraventricular arrhythmias including atrial fibrillation and reentrant tachyarrhythmias involving accessory pathways. Because amiodarone exhibits marked inter-individual variations, close monitoring of the individual is essential to adjust the amount of the drug delivered. The most important treatment-emergent adverse effects are hypotension, asystole/cardiac arrest/electromechanical dissociation (“EMD”), cardiogenic shock, congestive heart failure, bradycardia, liver function abnormalities, ventricular tachycardia, and atrio-ventricular block (See, e.g., Wyeth-Ayerst product insert for CORDARONE® Intravenous).

The solubility of amiodarone hydrochloride in water is low, but reportedly highly temperature dependent. The solubility ranges from 0.3 to 0.5 mg/ml at 20° C. to about 7 mg/ml at 50° C. At about 60° C., the solubility increases to greater than 100 mg/ml. At concentrations of about 50 mg/mL, amiodarone reportedly forms colloidal structures about 100 nm in diameter and micelles containing approximately 150 monomeric units and having a molecular weight in excess of 100,000 (Ravin et al., J. Pharm. Sci. (1975), 64 (11), 1830-1833).

Due to its low intrinsic water solubility, amiodarone is difficult to formulate in a water-based parenteral formulation that is sufficiently concentrated and stable and present in a medium having a physiologically acceptable pH. The water solubility can be increased by adjusting the pH of the solution to a value below its pKa, where the amiodarone takes on a positive ionic charge. Bonati et al. (J. Pharm. Sci. (1984), 73 (6), 829-831) report a pKa of 6.56. Even at low pH values, the solubility is insufficient to provide a ready to use formulation in the concentration range of 1-2 mg/mL.

Co-solvents and or surfactants can also be used to solubilize amiodarone in water. Ravin et al. (J. Pharm. Sci. (1969), 58 (10), 1242-45) report that cetyldimethyl-benzylammonium chloride, sodium lauryl sulfate and Tween® 80 increased the solubility of amiodarone at surfactant concentrations up to about 0.02% wt. Higher concentrations of sodium lauryl sulfate led to the formation of a colloidal suspension.

The currently marketed formulation (CORDARONE® Intravenous; CORDARONE I.V.®) of amiodarone contains 50 mg/ml amiodarone hydrochloride (“HCl”), 20.2 mg/mL benzyl alcohol and 100 mg/mL polysorbate 80 (TWEEN® 80; a nonionic surfactant, emulsifier, dispersant and/or stabilizer) in water. The CORDARONE I.V.® formulation is packaged in single use containers.

Polysorbate 80 and benzyl alcohol, however, are known to cause unwanted side effects. For example, polysorbate 80, either alone or in combination with benzyl alcohol, reportedly acts as a potent cardiac depressant and causes hypotension and cancer. Moreover, parenteral administration of benzyl alcohol has reportedly been associated with hemolysis, death and a number of other side effects.

A number of patents and scientific publications disclose parenteral preparations of amiodarone that reportedly have reduced side effects as compared to the currently marketed formulation. U.S. Pat. No. 5,234,949 to Ehrenpreis et al. discloses a parenteral solution of amiodarone (25-75 mg/ml) in a surfactant-free acetate buffer solution having a pH below 4. Ehrenpreis et al. disclose that the concentration and choice of the buffering agent are critical for physical stability in order to reduce precipitation or gel formation. Solutions containing amiodarone at concentrations of 15-50 mg/ml in an acetate buffer with a pH of between 3.2 and 3.8 cannot be diluted in glucose-saline water beyond 1 mg/ml without forming very opalescent or even milky solutions. Preparation of the 15-50 mg/mL formulations is reported to include a step whereby the solutions are heated to 60° C.-75° C. The preparations can be sterilized by filtration and sealed in sterile glass ampoules.

U.S. Pat. No. 6,143,778 to Gautier et al. discloses a parenteral formulation containing amiodarone, a buffer solution and a non-ionic hydrophilic surfactant. The hydrophilic surfactant is required in order avoid the above-mentioned problem associated with dilution of a buffered solution containing amiodarone hydrochloride. Solutions containing 1.5-8.0% wt. amiodarone were reportedly prepared in the presence of surfactant. Solutions containing 30-50 mg amiodarone/mL of solution at pH 2.4-3.8 were reportedly prepared in the presence of buffers such as acetate (0.1-0.3 M), phosphate (0.1-0.15 M), or glycine (0.2 M), where the ionic strength was maintained between 0.08-0.3 M. At higher ionic strengths, cloudy solutions were reported. Citrate reportedly was not suitable at any concentration. Suitable surfactants reportedly included nonionic hydrophilic compounds with HLB values in the range of 13-29, and present in concentrations of about 0.5-2.0%. Some stated examples were Pluronics®, Cremophors®, Tweens® and Solutols®. The formulation reportedly could be diluted to concentrations both approximating (about 0.5-0.8 mg/mL) and below (0.1-0.15 mg/mL) the critical concentration zone where turbidity is normally observed with dilution of aqueous amiodarone solutions prepared by heating.

Ravin et al. (J. Pharm. Sci. (1975), 64 (11), 1830-1833) disclose that chloride ion suppresses the solubility of amiodarone and that sodium citrate and tartrate, in very low concentrations ranging from 0.002-0.008 M and at pH values of 4.3-5.4, increase the solubility of amiodarone to 4.8 and 6 mg/mL, respectively. At higher concentrations, however, the solubility was suppressed. Under the conditions tested, acetate in any concentration decreased the solubility of amiodarone at pH 4-4.7. The ability to prepare more concentrated solutions of amiodarone was demonstrated to be temperature dependent. At 25° C., 40° C., and about 60° C., amiodarone concentrations of 0.35 mg/mL, 0.95 mg/mL and >13 mg/mL, respectively, could be achieved. The solution heated to 60° C. could be cooled to 25° C. without precipitation; however, it could not be diluted below the critical micellar concentration (“CMC”) without precipitation. The reported CMC value was approximately 0.5 mg/mL.

Contrary to the findings of Ravin et al., Benedini, et al. (J. Colloid Interface Sci. (2010), 342 (2), 407-414) report a CMC of 1.69 mg/mL occurring at a Krafft point of ˜70° C. and a pKa value for amiodarone of 7.95 in aqueous solution. They also describe a complicated phase diagram where the CMC value changes with temperature (increasing with temperatures below the Krafft point) and where at room temperature, a coacervate exists at amiodarone concentrations from the CMC down to about 0.8 mg/mL, and a gel forms in solutions diluted below this concentration.

Mosher et al. (U.S. Pat. No. 6,869,939) disclose aqueous formulations of amiodarone containing a sulfoalkyl ether cyclodextrin (“SAE-CD”). Formulations having a molar ratio of SAE-CD to amiodarone greater than or equal to 1.1:1 are reported to be dilutable in water without significant precipitation of amiodarone, i.e. if precipitation occurs it is less than or equal to about 3% wt. Adjusting the pH of the formulation reportedly can enhance the chemical stability in terms of precipitate or gel forming. Exemplified formulations are prepared as 50 mg/mL or greater concentrates of amiodarone which are prepared in cyclodextrin solutions at temperatures of 55° C. or 75° C. Dilution of a 50 mg/mL amiodarone formulation to ˜0.6-8.3 mg/mL with a dextrose solution is reported at room temperature. Formulations containing 50 or 0.5 mg/mL amiodarone were reportedly prepared in cyclodextrin solutions at room temperature and subsequently dried to prepare powders. The aqueous solubility of amiodarone in the presence of other derivatized cyclodextrins including 2-hydroxypropyl beta cyclodextrin is reported.

Cyclodextrins and their derivatives are widely used in liquid formulations to enhance the aqueous solubility of hydrophobic compounds. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds, which can fit all, or part of their structure into these cavities. This process, known as inclusion complexation, may result in increased apparent aqueous solubility and stability for the complexed drug. The complex is stabilized by hydrophobic interactions and does not involve the formation of any covalent bonds.

Chemical modification of the parent cyclodextrins (usually at the hydroxyl moieties) has resulted in substituted cyclodextrins with sometimes improved safety while retaining or improving the complexation ability of the cyclodextrin. Of the numerous substituted cyclodextrins prepared to date, two have been included in commercial injectable pharmaceutical formulations; the 2-hydroxypropyl derivatives (“HP-β-CD” or “HPCD”), neutral molecules being commercially developed by Janssen and others, and the sulfoalkyl ether derivatives (“SAE-β-CD” or “SAE-CD”), being developed by CyDex and others.

The SAE-CDs are a class of negatively charged cyclodextrins, which vary in the nature of the alkyl spacer, the salt form, the degree of substitution and the starting parent cyclodextrin. The presence of the negative charge allows for ionic interactions with drugs in solution as well as complexation.

Amiodarone is currently marketed and sold in vials, ampoules and syringes as a concentrate of 50 mg/mL amiodarone hydrochloride. The formulation is typically diluted with dextrose to a concentration of 0.5 to 2 mg/mL prior to administration. Since injectable forms of amiodarone are often used in emergency conditions (e.g. cardiac arrest), having a solution, premixed at the required dosing concentration, ready for administration is critical. The extra time required for preparation of a dilution can delay therapy and potentially have serious and negative consequences for the patient.

Several patents disclose amiodarone formulations that are reported to be ready to use. U.S. Pat. No. 7,067,143 to Doty et al report formulations at pH 2.9-3.2 containing 0.2 to 6 mg/mL amiodarone and a non-ionic surfactant in a polymeric container. Preparation of the formulation is exemplified with the steps of dissolving the surfactant, Tween® 80, in 40% of the final volume of water, heating the solution to 55° C., adding and dissolving the amiodarone, cooling the solution to 30° C., adding and dissolving dextrose, diluting to 90% of final volume, adjusting the pH to 3, then diluting to the final volume. The formulations can be sterilized and filled into plastic or other acceptable containers. When stored in plastic containers at 25° C., the formulations showed a loss of 1.6% amiodarone due to adsorption to the container and degradation of about 1% at 1 month, 1.5% at 4 months and 2.6% at 11 months.

U.S. Pat. No. 6,479,541 to Kipp et al report ready to use, surfactant free formulations of amiodarone containing a lactate and/or methanesulfonate buffer. Preparation of the formulation is described as including steps of dissolving amiodarone and a lactate and/or methanesulfonate buffer in 45-60° C. water, adjusting the pH to 3-4.5 and diluting the solution to a final volume. The formulations are chemically stable when refrigerated or frozen.

The inventors have identified improved ready to administer formulations containing amiodarone that are premixed at the concentrations recommended in the United States Food and Drug Administration (“FDA”) approved commercial labeling and typically used in clinical therapy, remain chemically and physically stable under a variety of storage conditions in both glass and plastic containers, and reduce the severity or occurrence of side effects, such as hypotension, bradycardia, hemolysis, and phlebitis, which are associated with the presently marketed formulations of amiodarone. Additionally, the inventors have identified a process for making the formulations that avoids the formation of gels and particulates, minimizes foaming, and that eliminates the need for a surfactant or organic solvent and therefore avoids side effects associated therewith. None of the art discloses or suggests the invention as claimed herein.

### III. SUMMARY OF THE INVENTION

The invention encompasses aqueous parenteral formulations including amiodarone or a salt thereof and a substituted cyclodextrin. The liquid formulations are isoosmotic, clear, sterile, and chemically and physically stable under a variety of storage conditions. The liquid formulations do not require a surfactant or organic solvent. The formulation includes a substituted cyclodextrin such as a sulfoalkyl ether cyclodextrin (“SAE-CD”), a hydroxyalkyl ether cyclodextrin (“HAE-CD”), a sulfoalkyl ether-alkyl ether cyclodextrin (“SAE-AE-CD”) or a sulfoalkyl ether-hydroxyalkyl ether cyclodextrin (“SAE-HAE-CD”) which provides significant advantages over other formulations of amiodarone. In certain embodiments, the liquid formulations of the invention are formulations included, for example, in an IV-bag, or bottle and are ready to use, requiring no further dilution or admixture.

In one embodiment, the invention encompasses a ready to use injectable intravenous bag formulation comprising a bag comprised of polyvinyl chloride, polyolefin, polypropylene, polyethylene, polyvinylidene chloride, nylon or combinations thereof comprising:
i. an aqueous solution containing a sulfobutylether beta-cyclodextrin and amiodarone or a pharmaceutically acceptable salt thereof wherein,
   1. the sulfobutylether beta-cyclodextrin:amiodarone mole ratio is greater than 2.7:1 and less than or equal to 7: 1; and
   2. the amiodarone concentration ranges from about 0.7 mM to about 7 mM.

The invention encompasses liquid formulations including amiodarone or a salt thereof complexed with a SAE-CD, a HAE-CD, a SAE-AE-CD, or a SAE-HAE-CD which are useful as an antiarrhythmic agent indicated for example, for the treatment and prophylaxis of frequently recurring ventricular fibrillation and hemodynamically unstable ventricular tachycardia in patients refractory to other therapy. The liquid formulations of the invention are used to suppress cardiac arrhythmias and/or life threatening arrhythmias. An arrhythmia can be suppressed in a patient by administering a therapeutically effective initial dose of a formulation of the invention including the amiodarone or salt thereof followed by a maintenance dose over a period of time sufficient to suppress the arrhythmia. In general, the initial loading dose of the amiodarone or salt thereof is accomplished by a first rapid infusion or injection of a therapeutically effective dose followed by a slow infusion or injection of a therapeutically effective dose depending on the needs of the individual patient. In certain embodiments, maintenance of an antiarrhythmic action with the formulations of the invention including amiodarone or salt thereof is typically accomplished by administering to a patient by injection or infusion of a lower amount of a therapeutically effective dose of the formulation of the invention including amiodarone or salt thereof over a period of time depending upon the individual needs of the patient.

The invention also encompasses methods of preparing ready to use aqueous parenteral formulations including amiodarone or a salt thereof and a substituted cyclodextrin. The methods substantially reduce or eliminate the formation of a gel in the solution. The methods provide a ready to use formulation with reduced potential for forming a foam when agitated or during manufacture and which can meet the United States Pharmacopeial requirements for particulate matter in injections upon-storage in pharmaceutically acceptable containers. The formulations are stable when stored under a variety of temperature conditions.

In one embodiment the invention encompasses a method for making a ready to use injectable pharmaceutical composition comprising the steps of:
a. preparing an aqueous solution containing a sulfobutylether beta-cyclodextrin and amiodarone or a pharmaceutically acceptable salt thereof, wherein:
   i. the sulfobutylether beta-cyclodextrin:amiodarone mole ratio is greater than 2.7:1 and less than or equal to 7:1, and
   ii. the amiodarone is dissolved while maintaining a solution temperature ranging from about 15° C. to about 65° C. and a pH less than or equal to the pKa of amiodarone to give a clear solution absent of gel,
b. adding water as needed to provide a final solution with an amiodarone concentration ranging from about 0.7 mM to about 7 mM;
c. sterile filtering the final solution to substantially reduce microbial contamination; and
d. aseptically filling the final solution into a pharmaceutically acceptable container.

In another embodiment the invention encompasses a method for making a ready to use injectable pharmaceutical composition free of visible particulates comprising the steps of:
a. preparing an aqueous solution comprising a sulfobutylether-beta-cyclodextrin and amiodarone or a pharmaceutically acceptable salt thereof, wherein:
   i. the sulfobutylether-beta-cyclodextrin:amiodarone mole ratio ranges from about 1.1:1 to less than or equal to about 7:1,
   ii. the amiodarone is dissolved while maintaining a solution temperature ranging from about 15° C. to less than about 40° C. and a pH less than or equal to the pKa of amiodarone to give a clear solution absent of gel,
b. optionally adding additional cyclodextrin as needed to provide a sulfobutylether-beta-cyclodextrin:amiodarone mole ratio ranging from greater than 2.7:1 to about 7:1; and
c. adding additional water as needed to provide a final solution with an amiodarone concentration ranging from about 0.73 mM to about 7.3 mM.

In another embodiment the invention encompasses a method for making a ready to use injectable pharmaceutical composition free of visible particulates comprising the steps of:
a. preparing an aqueous solution containing a sulfobutylether beta-cyclodextrin and amiodarone or a pharmaceutically acceptable salt thereof, wherein:
   i. the sulfobutylether-beta-cyclodextrin:amiodarone mole ratio ranges from about 1.5:1 to about 7:1,
   ii. the amiodarone is dissolved while maintaining a solution temperature ranging from about 15° C. to about 65° C. and a pH less than or equal to the pKa of amiodarone to give a clear solution absent of gel,
b. optionally adding additional sulfobutylether beta-cyclodextrin as necessary to provide a cyclodextrin:amiodarone mole ratio of greater than about 2.7:1 to about 7:1 in the final solution, and
c. adding additional water as needed to provide a final solution with an amiodarone concentration ranging from about 0.73 mM to about 7.3 mM.

In another embodiment the invention encompasses a method for making a ready to use injectable pharmaceutical composition free of visible particulates comprising the steps of:
a. preparing a concentrated aqueous solution containing sulfobutylether beta-cyclodextrin and amiodarone or a pharmaceutically acceptable salt thereof, wherein:
   i. the volume of the solution represents from about 1% to less than about 17% of the volume of the final solution,
   ii. the sulfobutylether-beta-cyclodextrin:amiodarone mole ratio ranges from about 1.1:1 to about 7:1,
   iii. the amiodarone is dissolved while maintaining a solution temperature of about 15° C. to about 65° C. and a pH less than or equal to the pKa of amiodarone to give a solution absent of gel,
b. optionally adding additional sulfobutylether beta-cyclodextrin as necessary to provide a final solution with a cyclodextrin:amiodarone mole ratio of greater than about 2.7:1 to about 7:1, and
c. adding additional water, as necessary, to provide a final solution with an amiodarone concentration ranging from about 0.73 mM to about 7.3 mM.

In another embodiment the invention encompasses a method for making a ready to use injectable pharmaceutical composition free of visible particulates comprising the steps of:
a. preparing a first aqueous solution containing sulfobutylether beta-cyclodextrin, amiodarone or a pharmaceutically acceptable salt thereof, and optionally containing one or more components selected from pH adjusting agents, buffering agents, tonicity modifying agents and antioxidants, wherein:
   i. the volume of the first aqueous solution represents from about 1% to less than about 17% of the volume of the final solution,
   ii. the sulfobutylether-beta-cyclodextrin:amiodarone mole ratio ranges from about 1.1:1 to about 7:1,
   iii. the amiodarone is dissolved while maintaining a solution temperature of about 15° C. to about 65° C. and a pH less than or equal to the pKa of amiodarone to give a clear solution absent of gel,
b. preparing a second aqueous solution containing one or more components selected from pH adjusting agents, buffering agents, tonicity modifying agents, antioxidants, and additional sulfobutylether beta-cyclodextrin,
c. combining the two solutions and mixing the resulting solution to give a clear solution absent of gel, and
d. optionally adding additional water and sulfobutylether beta-cyclodextrin as necessary to provide a final solution with an amiodarone concentration of about 0.73 mM to about 7.3 mM and a cyclodextrin:amiodarone mole ratio ranging from greater than about 2.7:1 to about 7:1.

In another embodiment the invention encompasses a method for making a ready to use pharmaceutical composition free of visible particulates comprising the steps of:
a. adding a volume of water for injection equivalent to 20% to 30% of the final solution volume to a suitable container and adjusting the temperature to 20° C.-30° C.,
b. adding and dissolving sulfobutyl ether-7-beta-cyclodextrin, citric acid and sodium citrate such that the solution pH is in the range of about 3 to about 4,
c. adding and dissolving amiodarone hydrochloride in an amount sufficient to provide a cyclodextrin:amiodarone mole ratio in the final solution ranging from about 3.1:1 to 3.2:1 to give a clear solution absent of gel,
d. adding additional water for injection to bring the solution volume to 70%-80% of the final solution volume, and dissolving sufficient dextrose to provide a final solution osmolality of about 255 mOsm/kg to about 345 mOsm/kg,
e. adjusting the pH to 3.55-3.65 with hydrochloric acid or sodium hydroxide,
f. adding additional water for injection to bring the solution to a final solution volume wherein the amiodarone concentration ranges from about 2 mM to 3 mM and the citrate concentration ranges from about 2 mM to 3 mM,
g. sterile filtering the solution to substantially reduce microbial contamination, and
h. aseptically filling the solution into a pharmaceutically acceptable container.

### IV. BRIEF DESCRIPTION OF THE DRAWINGS

FIG. 1 illustrates the solubility of amiodarone hydrochloride in saline solutions containing 5 mM citrate buffer at pH 3.6 and sulfobutylether β-cyclodextrin, (□ 9 mg/mL sodium chloride; ♦ 7 mg/mL sodium chloride).

FIG. 2 illustrates the solubility of amiodarone hydrochloride in water in the presence of increasing amounts of sodium chloride.

FIG. 3 illustrates the solubility of amiodarone in the presence of 8.1 mg/mL sulfobutylether beta cyclodextrin and increasing amounts of a citrate buffer at a pH of about 3.5.

FIG. 4 illustrates the solubility of amiodarone hydrochloride in a 5% dextrose solution containing 0.9 mM citrate buffer at pH 3.8 and sulfobutylether β-cyclodextrin.

FIG. 5 illustrates the solubility of amiodarone hydrochloride in water adjusted to pH 4.5 and containing 2-hydroxypropyl β-cyclodextrin having different degrees of substitution, (▪ HP-7 β-cyclodextrin; □ HP-4 β-cyclodextrin).

### V. DETAILED DESCRIPTION OF THE INVENTION

Unless otherwise specified, all percentages and amounts expressed herein and elsewhere in the specification should be understood to refer to percent by weight. The concentration is denoted in mg/mL. Also, the term “about,” when used in reference to a range of values, should be understood to refer to either value in the range, or to both values in the range.

As used throughout, ranges are used as shorthand for describing each and every value that is within the range. Any value within the range can be selected as the terminus of the range.

All documents, for example, scientific publications, patents, patent applications and patent publications, recited herein are hereby incorporated by reference in their entirety to the same extent as if each individual document was specifically and individually indicated to be incorporated by reference in its entirety. In the event of a conflict in a definition in the present disclosure and that of a cited reference, the present disclosure controls.

### Definitions

As used herein, the term “alkalizing agent” is intended to mean a compound used to provide an alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

The terms “alkylene” and “alkyl,” as used herein (e.g., in the —O—(C₂-C₆-alkylene) SO₃⁻ group or in the alkylamines), include linear, cyclic, and branched, saturated and unsaturated (i.e., containing one double bond) divalent alkylene groups and monovalent alkyl groups, respectively. The term “alkanol” in this text likewise includes linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

As used herein, the term “antioxidant” is intended to mean an agent that inhibits oxidation and thus is used to prevent the deterioration of preparations by the oxidative process. Such compounds include, by way of example and without limitation, acetone, potassium metabisulfite, potassium sulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid and sodium metabisulfite and others known to those of ordinary skill in the art.

As used herein, the term “buffering agent” is intended to mean a compound used to resist change in pH upon storage, dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, sodium acetate, sodium bicarbonate, sodium tartrate and sodium citrate anhydrous and dihydrate and others known to those of ordinary skill in the art.

As used herein, the term “gel” is intended to mean a colloidal dispersion of particles that forms a solid or semisolid. For example, gels are visible area(s) or region(s) of a solution that appear as an undissolved semisolid mass. Additional non-limiting information on gels can be found in the following references: Remington, The Science and Practice of Pharmacy, 21^{st} Edition, and The United States Pharmacopoeia 30, 2007, Chapter <1151>.

By “complexed” is meant “being part of a clathrate or inclusion complex with”, i.e., a complexed therapeutic agent is part of a clathrate or inclusion complex with a substituted cyclodextrin. By “major portion” is meant at least about 50% by weight of the therapeutic compound. The actual percent of drug that is complexed will vary according to the complexation equilibrium constant characterizing the complexation of a specific cyclodextrin derivative to a specific drug and to the concentrations of the cyclodextrin derivative and drug available for complexation. The complexation constant can be determined experimentally by conducting phase solubility studies (Higuchi, T. and Connors, K. A. in “Advances in Analytical Chemistry and Instrumentation Vol. 4” Reilly, Charles N. Ed., John Wiley & Sons., 1965, pp. 117-212) where the solubility of a drug is determined in the presence of increasing amounts of a cyclodextrin or substituted cyclodextrin.

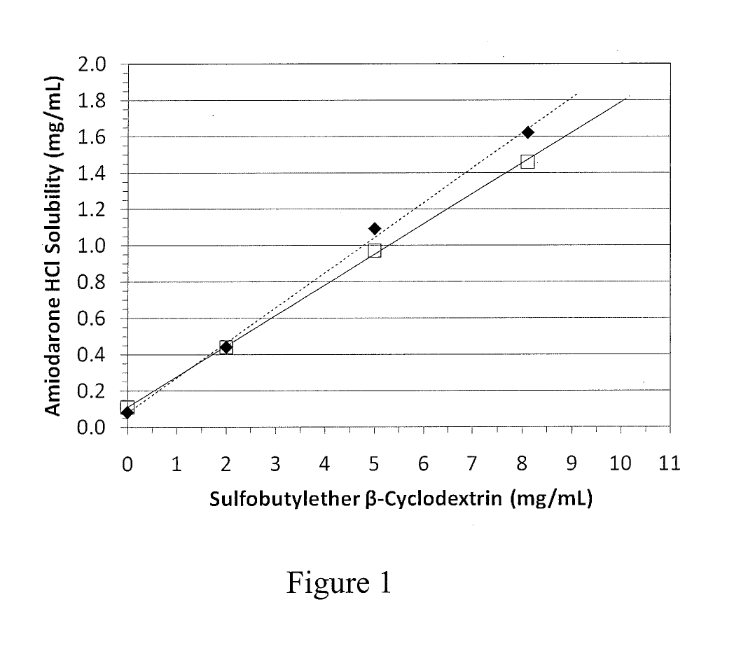

Phase solubility studies can also generate information as to the required amounts of a cyclodextrin needed to solubilize a drug under different conditions such as temperature, cyclodextrin type, or solution composition. For example, FIG. 1 depicts a phase solubility curve for SBE7-β-CD and amiodarone at pH 3.6 in a solution containing a 5 mM citrate buffer and sodium chloride at 7 or 9 mg/mL and at about 25° C. The figure shows that as the sodium chloride is increased from 7 to 9 mg/mL, the complexation decreases and more cyclodextrin is required to reach the same concentration of amiodarone. A solution containing 1.8 mg/mL amiodarone hydrochloride in 9 mg/mL sodium chloride should require about 10 mg/mL SBE7-β-CD to provide complete solubilization of the amiodarone.

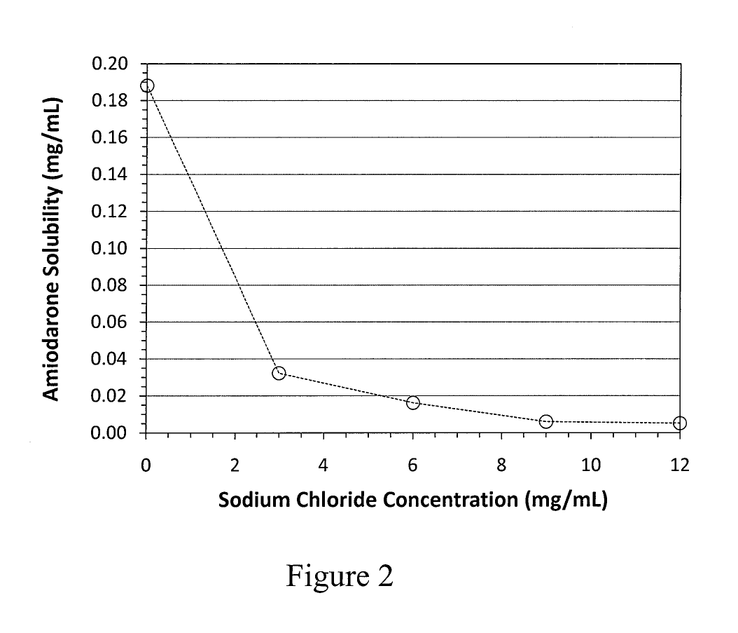

The presence of sodium chloride can affect the solubility of amiodarone in the absence of a cyclodextrin as depicted in FIG. 2. At pH values below the pKa of amiodarone, a positive charge is present on the amiodarone molecule. This promotes increased water solubilization. However, as the ionic strength is increased, such as occurs with the addition of sodium chloride, this solubilization is suppressed. In addition, if a hydrochloride salt form of amiodarone is being evaluated, a common ion effect can be observed with the added chloride suppressing solubilization.

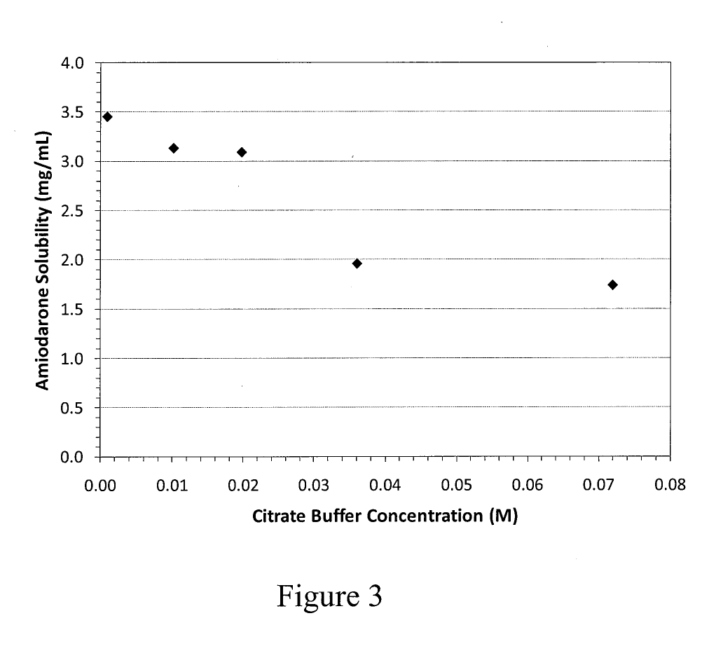

FIG. 3 illustrates a similar effect with added buffer species. The negatively charged sulfoalky ether cyclodextrin solubilizes amiodarone both by complexation and by ionic interactions. Increasing the amount of ionic buffer present in the solution decreases the solubilization of amiodarone in the water and also decreases the solubilization by the negatively charged cyclodextrin.

At pH values where amiodarone is positively charged, it is expected that substituted cyclodextrins containing negatively charged functional groups will solubilize amiodarone to a greater extent than neutral cyclodextrins, and the solubilization will vary with the number of charged substituents. The presence of other components in the formulation, especially charged ionic components will have an effect on the solubilization by either charged or neutral substituted cyclodextrins.

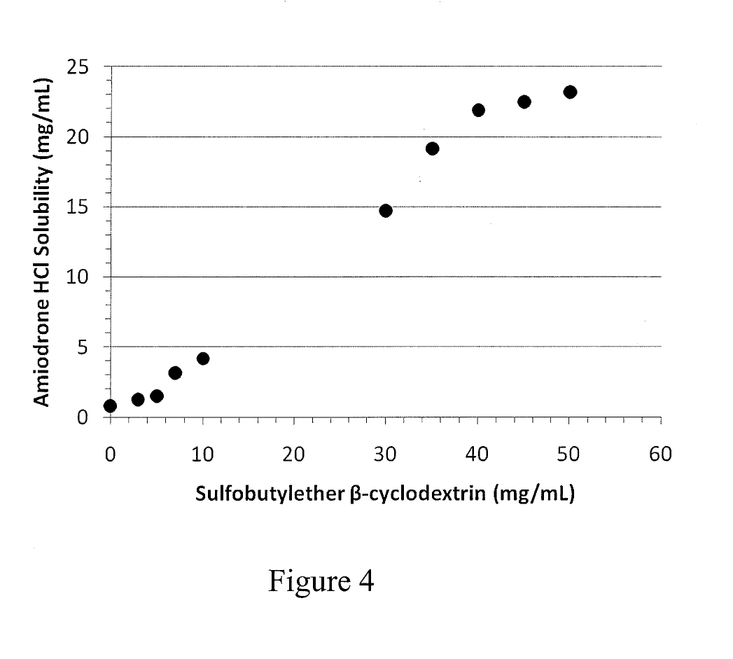

A 5% dextrose solution provides a comparable tonicity to 9 mg/mL saline solution, but without the ionic charges that are present with the sodium chloride. FIG. 4 depicts a room temperature phase solubility curve for SBE7-β-CD and amiodarone in a solution containing dextrose 5% and 0.9 mM citrate buffer at pH 3.8. A solution containing 1.8 mg/mL amiodarone hydrochloride in 5% dextrose solution should require about 6 mg/mL SBE7-β-CD. Amiodarone shows greater solubilization by SBE7-β-CD in the presence of dextrose than in the presence of sodium chloride.

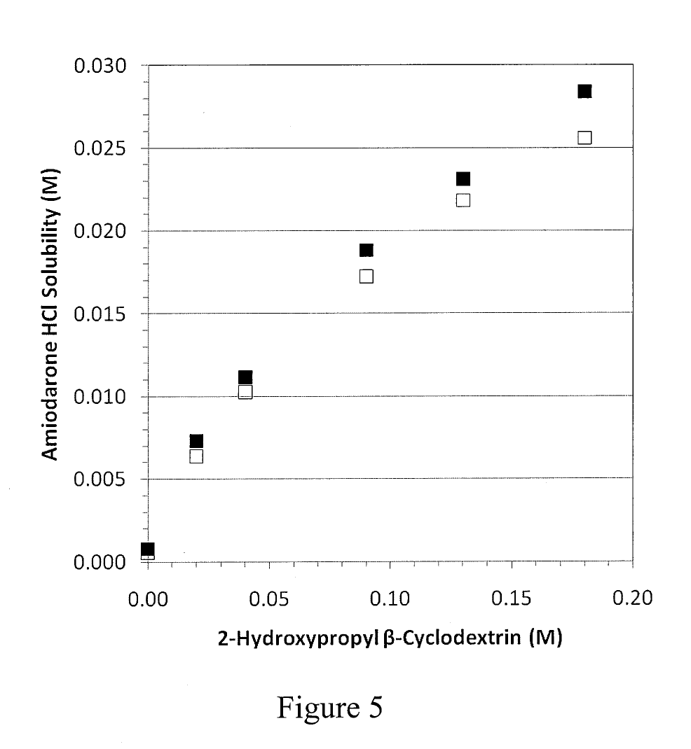

Other substituted cyclodextrins also form complexes with amiodarone. The phase solubility of amiodarone hydrochloride in water at pH 4.5 in the presence of 2-hydroxypropyl β-cyclodextrin having an average of about 7 or 4 hydroxypropyl substituents (actual DS of 7.6 and 4.3 with molecular weights 1576 and 1384 g/m respectively) on the cyclodextrin ring is shown in FIG. 5. These HAE-CDs both solubilize amiodarone, the SBE7-β-CD solubilizing slightly more drug than the SBE4-β-CD derivative. A 1.8 mg/mL amiodarone hydrochloride solution requires about 9 mg/mL SBE4-β-CD for solubilization in the absence of other components such as tonicity modifying agents or buffering agents.

A “complexation-enhancing agent” can be added to the aqueous liquid formulation of the invention. A complexation-enhancing agent is a compound, or compounds, that enhance(s) the complexation of amiodarone with the SAE-CD, HAE-CD, SAE-AE-CD or SAE-HAE-CD. When the complexation-enhancing agent is present, the required ratio of substituted-CD to amiodarone may need to be changed such that less cyclodextrin is required. Suitable complexation enhancing agents include one or more pharmacologically inert water soluble polymers, hydroxy acids, and other organic compounds typically used in liquid formulations to enhance the complexation of a particular agent with cyclodextrins. Suitable water soluble polymers include water soluble natural polymers, water soluble semisynthetic polymers (such as the water soluble derivatives of cellulose) and water soluble synthetic polymers. The natural polymers include polysaccharides such as inulin, pectins, algin derivatives and agar, and polypeptides such as casein and gelatin. The semi-synthetic polymers include cellulose derivatives such as methylcellulose, hydroxyethyl cellulose, hydroxypropyl cellulose, their mixed ethers such as hydroxypropyl methylcellulose and other mixed ethers such as hydroxyethyl ethylcellulose, hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Suitable hydroxy acids include by way of example, and without limitation, citric acid, malic acid, lactic acid, and tartaric acid and others known to those of ordinary skill in the art.

The liquid formulation of the invention will comprise an effective amount of amiodarone or a salt thereof The term “effective amount” refers a therapeutically effective amount is contemplated. A therapeutically effective amount is an amount or quantity of amiodarone or a salt thereof that is sufficient to elicit the required or desired therapeutic response or an amount that is sufficient to elicit an appreciable biological response when administered to a subject.

As used herein and unless otherwise indicated, the term “formulations of the invention” means a liquid formulation for parenteral administration especially for intravenous administration which includes amiodarone or a salt thereof, a substituted cyclodextrin such as a SAE-CD, a HAE-CD, a SAE-AE-CD or a SAE-HAE-CD and one or more carriers. Illustrative formulations encompassed by the term “formulations of the invention” are described herein.

As used herein, the term “injectable pharmaceutical composition” refers to a composition suitable for administration to a patient or subject that is essentially free of visible particulates, for example, a composition meeting the requirements of United States Pharmacopeia 33, Chapter <1>Injections.

As used herein the term “non-covalent ionic bond” refers to a bond formed between an anionic species and a cationic species. The bond is non-covalent such that the two species together form a salt or ion pair. The SAE-CD, SAE-AE-CD or SAE-HAE-CD provides the anionic species of the ion pair and the amiodarone provides the cationic species of the ion pair. Since the SAE-CD, SAE-AE-CD and SAE-HAE-CD are multi-valent, they can form an ion pair with one or more cationic amiodarone species.

As used herein, the term “patient” or “subject” refers to warm blooded animals such as mammals, for example, cats, dogs, mice, guinea pigs, horses, bovine cows, sheep, and humans.

As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, a “pharmaceutically acceptable liquid carrier” is any aqueous medium used in the pharmaceutical sciences for dilution or dissolution of parenteral formulations. In a specific embodiment, the term “pharmaceutically acceptable” means generally accepted by or approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a formulation of the invention is administered. Such pharmaceutical carriers can be liquids, such as water, saline, aqueous solutions and the like. When administered to a patient, the formulations of the invention and pharmaceutically acceptable vehicles are preferably sterile. Water is a preferred vehicle when the compound of the invention is administered intravenously. Saline solutions and aqueous dextrose solutions can also be employed as liquid vehicles, particularly for injectable solutions. The present compositions, if desired, can also contain minor amounts of wetting agents, or pH buffering agents.

As used herein, the term “pharmaceutically acceptable container” is intended to mean a container closure system that: protects the drug product, for example, from factors that can cause degradation of the dosage form over its shelf-life; is compatible with the drug product, for example, the packaging components will not interact sufficiently to cause unacceptable changes in the quality of either the drug or the packaging component, such as absorption or adsorption of the drug substance, degradation of the drug substance that is induced by extractables/leachables from the container, precipitation, and changes in pH; and is safe, for example, a container that does not leach harmful or undesirable amounts of substances to which a patient will be exposed when being treated with the product, or in the case of injectable formulations, the container will protect the formulation from the introduction of microbes and not contain pyrogens. Containers useful for injectable formulations are often sterilized prior to and/or after being filled with the formulation. Pharmaceutically acceptable containers include, but are not limited to, intravenous bags, bottles, vials and syringes. Suitable pharmaceutically acceptable containers include an evacuated container, a syringe, bag, pouch, ampoule, vial, bottle, or any pharmaceutically acceptable device known to those skilled in the art for the delivery of liquid formulations. Preferred containers are plastic or polymeric containers constructed from plastics such as polyamide, ethylene vinyl acetate, polyolefin, polypropylene, polyethylene, polyvinylidene chloride, nylon, and/or polyvinylchloride or combinations thereof and marketed under trade names such as GALAXY®, INTRAVIA®, AVIVA®, and VIAFLEX®. These containers are disclosed in U.S. Pat. No. 4,686,125, No. 4,692,361, No. 4,779,997, No. 5,849,843, No. 5,998,019, and No. 6,168,862. These containers can be comprised of a single type of plastic or polymer, a blend of plastics and or polymers, or a laminate of one or more different types of plastics and or polymers. It is contemplated, however, that most plastic containers will produce comparable results.

As used herein the term “pH adjusting agent” is an agent to increase or decrease the desired pH of the formulation when admixed into the formulation. The pH of the liquid formulation will generally range from about pH 3.0 to about pH 7.0; however, liquid formulations having higher or lower pH values can also be prepared. It is contemplated that amiodarone chemical and physical stability can be increased by optimizing the pH as well as the mole ratio of substituted cyclodextrin to amiodarone. Preferably the pH of the formulation ranges from pH 3 to about pH 4, and most preferably the pH of the formulation is 3.6. The pH of the composition may be adjusted using an appropriate pH adjusting agent, such as a suitable acid, base, amine, or any combination thereof. Preferably, a pH adjusting agent used in the formulation include hydrochloric acid, sodium hydroxide, amines, ammonium hydroxide, nitric acid, phosphoric acid, sulfuric acid, citric acid, organic acids, and/or salts thereof, and any combination thereof.

As used herein the term substituted cyclodextrin refers to an alpha, beta or gamma cyclodextrin having one or more of the hydroxyl groups in its structure replaced with a different chemical substituent attached through an ether linkage. The substituted cyclodextrin can contain a single type of chemical substituent or more than one type within the same cyclodextrin molecule. For example, a cyclodextrin can have one hydroxyl substituted with a sulfoalkyl substituent and another hydroxyl substituted with a hydroxyalkyl substituent. Substituted cyclodextrin compounds include, by way of example and without limitation, sulfoalkyl ether cyclodextrins, hydroxyalkyl ether cyclodextrins, sulfoalkyl ether-hydroxyalkyl ether cyclodextrins, sulfoalkylether-alkyl ether cyclodextrins and others known to those of ordinary skill in the art.

The number of hydroxyls that have been replaced in a cyclodextrin is represented by a number referred to as the degree of substitution (“DS”). It should be noted that preparation of substituted cyclodextrins occurs in a controlled, although not exact manner. For this reason, the degree of substitution is actually a number representing the average number of substituent groups per cyclodextrin (for example, SBE7-β-CD, has an average of 7 sulfobutyl ether substitutions per beta (β) cyclodextrin and SBE4-β-CD has an average of 4 hydroxypropyl substitutions). In addition, the regiochemistry of substitution of the hydroxyl groups of the cyclodextrin is variable with regard to the substitution of specific hydroxyl groups of the hexose ring. For this reason, substitution of different hydroxyl groups is likely to occur during manufacture of the substituted cyclodextrin, and a particular substituted cyclodextrin will possess a preferential, although not exclusive or specific, substitution pattern.

As used herein the term “sulfoalkyl ether cyclodextrin” (“SAE-CD”) refers to compounds encompassed by the formula 1:

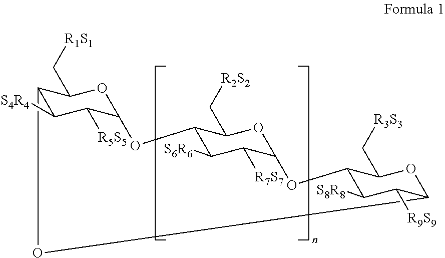

wherein n is 4, 5 or 6; R₁, R₂, R₃, R₄, R₅, R₆, R₇, R₈, and R₉ are each, independently, —O— or a —O—(C₂-C₆ alkylene)—SO₃⁻ group, wherein at least one of R₁ and R₂ is independently a —O—(C₂-C₆ alkylene)—SO₃⁻ group, preferably a —O—(CH₂)ₘSO₃− group, wherein m is 2 to 6, preferably 2 to 4, (e.g. —OCH₂CH₂CH₂SO₃⁻ or —OCH₂CH₂CH₂CH₂SO₃); and S₁, S₂, S₃, S₄, S₅, S₆, S₇, S₈ and S₉ are each, independently, a pharmaceutically acceptable cation which includes, for example, H⁺, alkali metals (e.g., Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine and (C₄-C₈)-cycloalkanolamine. In certain illustrative embodiments, n is 5; R₁, R₂, R₃, R₄, R₅, R₆, R₇, R₈, and R₉ are each —O— or —O—((CH₂)₄)—SO₃⁻; at least one of R₁ and R₂ is independently a —O—((CH₂)₄)—SO₃⁻ group; and S₁, S₂, S₃, S₄, S₅, S₆, S₇, S₈, and S₉ are each H or Na³⁰ . In certain embodiments, the SAE-CD is represented by formula 2:

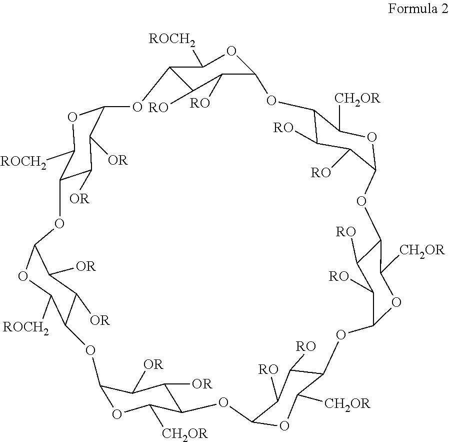

wherein R=H₂₁₋ₙ or (CH₂CH₂CH₂CH₂SO₂ONa)ₙ and where n=6.0-7.1. In certain illustrative embodiments, the sulfoalkyl ether cyclodextrin (“SAE-CD”) is sulfobutylether 7-beta-cyclodextrin.

As used herein the “hydroxyalkyl ether cyclodextrin” (“HAE-CD”) refers to compounds encompassed by the formula 3:

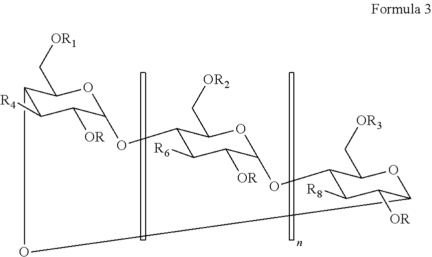

wherein n is 4, 5 or 6; R₁, R₂, R₃, R₄, R₅, R₆, R₇, R₈ and R₉ are each, independently, —H or a —(C₂-C₆ alkylene) group further substituted with at least one (—OH), wherein at least one of R₁ and R₂ is independently a —(C₂-C₆ alkylene) group further substituted with at least one (—OH). In certain illustrative embodiments, n is 5; R₁, R₂, R₃, R₄, R₅, R₆, R₇, R₈, and R₉ are each —H or —CH₂CH(OH)CH₃; and at least one of R₁ and R₂ is independently a —CH₂CH(OH)CH₃ group. In certain embodiments, the HAE-CD is represented by formula 4:

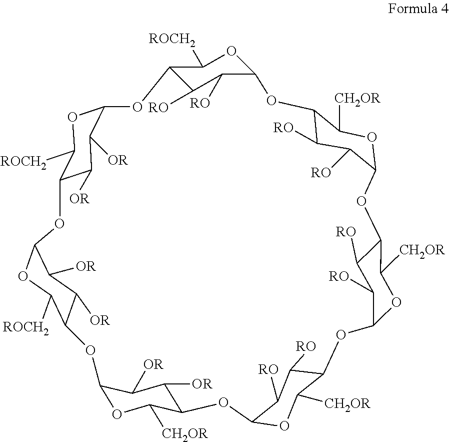

wherein R=H₂₁₋ₙ or (CH₂CHOHCH₃)ₙ and where n=4 or 6. In certain illustrative embodiments, the hydroxyalkyl ether cyclodextrin (“HAE-CD”) is 2-hydroxypropyl 4-beta-cyclodextrin.

As used herein, the term “tonicity modifying agent” is intended to mean a compound or compounds that can be used to adjust the tonicity of the liquid formulation. Suitable tonicity modifying agents include glycerin, lactose, mannitol, dextrose, sodium chloride, sodium sulfate, sorbitol, trehalose and others known to those of ordinary skill in the art. In one embodiment, the tonicity of the liquid formulation approximates the tonicity of blood or plasma. An isotonic or isoosmotic solution is one where the tonicity approximates the tonicity of blood or plasma.

As used herein, the term “visible particulates” is intended to mean particulate matter that is visible to the eye of a person trained in making such observations without the use of magnification. In some procedures for visual examination for particulates, the bag or container is swirled or gently mixed prior to examination. Solutions with a sufficiently low surface tension may foam during this step making the observation difficult or prone to false positive results. Such solutions can also foam during manufacture or handling prior to the visual examination and residual foam may be present during the examination, causing the same difficulties in observation and false results.

### General Description

The invention generally encompasses a ready to use injectable intravenous bag formulation comprising:

a. a bag comprising of polyvinyl chloride, polyolefin, polypropylene, polyethylene, polyvinylidene chloride, nylon, or combinations thereof comprising:
i. an aqueous solution containing a sulfobutylether beta-cyclodextrin and amiodarone or a pharmaceutically acceptable salt thereof wherein,
   1. the cyclodextrin:amiodarone mole ratio is greater than 2.7:1 and less than or equal to 7:1; and
   2. the amiodarone concentration ranges from about 0.7 mM to about 7 mM.

In certain embodiments, the solution is sterile.

In certain embodiments, the bag is comprised of ethylene vinyl acetate, polyolefin, polypropylene, polyethylene, nylon, and/or polyvinylchloride or combinations thereof.

In certain embodiments, the intravenous bag comprises polyvinyl chloride.

In certain embodiments, the intravenous bag comprises polyolefin.

In certain embodiments, the intravenous bag comprises ethylene vinyl acetate.

In certain embodiments, the intravenous bag comprises polypropylene.

In certain embodiments, the intravenous bag comprises polyethylene.

In certain embodiments, the intravenous bag comprises polypropylene.

In certain embodiments, the intravenous bag comprises a combination of one or more of ethylene vinyl acetate, polyolefin, polypropylene, polyethylene, nylon, and/or polyvinylchloride.

In certain embodiments, the intravenous bag formulation further comprises one or more components selected from pH adjusting agents, buffering agents, antioxidants, and tonicity modifying agents.

In certain embodiments, the aqueous solution contains about 0.9 mM to about 5 mM citrate buffering agent.

In certain embodiments, the cyclodextrin:amiodarone mole ratio ranges from about 2.9:1 to about 5:1.

In certain embodiments, the final solution sulfobutylether beta-cyclodextrin concentration ranges from about 13 mg/mL to about 40 mg/mL, and the amiodarone concentration ranges from about 2.2 mM to about 2.7 mM.

In certain embodiments, the final solution sulfobutylether beta-cyclodextrin concentration ranges from about 6 mM to about 18 mM, and the amiodarone concentration ranges from about 2.2 mM to about 2.7 mM.

In certain embodiments, the surface tension of the final solution ranges from about 59 dynes/cm to about 75 dynes/cm.

In certain embodiments, the aqueous solution has a pH of about 3.6, and comprises about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the aqueous solution has a pH of about 3.6 and comprises about 3 mM citrate buffer, about 2.6 mM amiodarone, about 8.3 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is formation of no more than about 0.1% (w/w) total impurities in the solution in the container, when stored at room temperature for 6 months.

In certain embodiments, there is no more than about 1% absorptive loss of amiodarone from the solution in the container, when stored at room temperature for 6 months.

In another embodiment, the invention generally encompasses a method for making a ready to use injectable pharmaceutical composition comprising the steps of:

a. preparing an aqueous solution containing a sulfobutylether beta-cyclodextrin and amiodarone or a pharmaceutically acceptable salt thereof wherein,
i. the cyclodextrin:amiodarone mole ratio is greater than 2.7:1 and less than or equal to 7:1, and
ii. the amiodarone is dissolved while maintaining a solution temperature of about 15° C. to about 65° C. and a pH less than or equal to the pKa of amiodarone, to give a clear solution absent of gel,

b. adding water as needed to provide a final solution with an amiodarone concentration of about 0.7 to 7 mM;

c. sterile filtering the final solution to substantially reduce microbial contamination; and

d. aseptically filling the final solution into a pharmaceutically acceptable container.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% degradation or absorptive loss of amiodarone from the solution in the container, when stored at room temperature for 6 months.

In certain embodiments, there is formation of no more than about 0.1% (w/w) total impurities in the solution in the container, when stored at room temperature for 6 months.

Another embodiment encompasses a method for making a ready to use injectable pharmaceutical composition free of visible particulates comprising the steps of:

a. preparing an aqueous solution comprising a sulfobutylether-beta-cyclodextrin and amiodarone or a pharmaceutically acceptable salt thereof wherein,
i. the cyclodextrin:amiodarone mole ratio ranges from about 1.1:1 to less than or equal to about 7:1,
ii. the amiodarone is dissolved while maintaining a solution temperature of about 15° C. to less than about 40° C. and a pH less than or equal to the pKa of amiodarone, to give a clear solution absent of gel,

b. optionally adding additional cyclodextrin as needed to provide a cyclodextrin:amiodarone mole ratio of greater than 2.7:1 to about 7:1; and

c. adding additional water as necessary to provide a final solution with an amiodarone concentration of about 0.73 mM to about 7.3 mM.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% absorptive loss of amiodarone from the solution in the container, when stored at room temperature for 6 months.

In certain embodiments, there is formation of no more than about 0.1% (w/w) total impurities in the solution in the container, when stored at room temperature for 6 months.

In certain embodiments, the final solution is sterile filtered to substantially reduce the microbial contamination and the filtered solution is aseptically filled into a pharmaceutically acceptable container.

In certain embodiments, dextrose is added to provide a final solution osmolality of about mOsm/kg 255 to about 345 mOsm/kg.

In certain embodiments, sodium chloride is added to provide a final solution osmolality of about 255 mOsm/kg to about 345 mOsm/kg.

In certain embodiments, there is formation of no more than about 0.1% (w/w) total impurities in the solution in the container, when stored at room temperature for 6 months.

Another embodiment encompasses a method for making a ready to use injectable pharmaceutical composition free of visible particulates comprising the steps of:

a. preparing an aqueous solution containing a sulfobutylether beta-cyclodextrin and amiodarone or a pharmaceutically acceptable salt thereof wherein,
i. the cyclodextrin:amiodarone mole ratio ranges from about 1.5:1 to about 7:1,
ii. the amiodarone is dissolved while maintaining a solution temperature of about 15° C. to about 65° C. and a pH less than or equal to the pKa of amiodarone, to give a clear solution absent of gel,

b. optionally adding additional sulfobutylether beta-cyclodextrin as necessary to provide a cyclodextrin:amiodarone mole ratio of greater than about 2.7:1 to about 7:1 in the final solution, and

c. adding additional water as necessary to provide a final solution with an amiodarone concentration of 0.73 to 7.3 mM.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% absorptive loss of amiodarone from the solution in the container, when stored at room temperature for 6 months.

In certain embodiments, there is formation of no more than about 0.1% (w/w) total impurities in the solution in the container, when stored at room temperature for 6 months.

Another embodiment encompasses a method for making a ready to use injectable pharmaceutical composition free of visible particulates comprising the steps of:

a. preparing a concentrated aqueous solution containing sulfobutylether beta-cyclodextrin and amiodarone or a pharmaceutically acceptable salt thereof wherein,
i. the volume of the solution represents from about 1% to less than about 17% of the volume of the final solution,
ii. the cyclodextrin:amiodarone mole ratio ranges from about 1.1:1 to about 7:1,
iii. the amiodarone is dissolved while maintaining a solution temperature of about 15° C. to about 65° C. and a pH less than or equal to the pKa of amiodarone, to give a solution absent of gel,

b. optionally adding sulfobutylether beta-cyclodextrin as necessary to provide a final solution with a cyclodextrin:amiodarone mole ratio of greater than about 2.7:1 to about 7:1, and

c. adding additional water, as necessary, to provide a final solution with an amiodarone concentration of 0.73 to 7.3 mM.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% absorptive loss of amiodarone from the solution in the container, when stored at room temperature for 6 months.

In certain embodiments, there is formation of no more than about 0.1% (w/w) total impurities in the solution in the container, when stored at room temperature for 6 months.

Another embodiment encompasses a method for making a ready to use injectable pharmaceutical composition free of visible particulates comprising the steps of:

a. preparing a first aqueous solution containing sulfobutylether beta-cyclodextrin, amiodarone or a pharmaceutically acceptable salt thereof, and optionally containing one or more components selected from pH adjusting agents, buffering agents, tonicity modifying agents and antioxidants wherein,
i. the volume of the first aqueous solution represents from about 1% to less than about 17% of the volume of the final solution,
ii. the cyclodextrin:amiodarone mole ratio ranges from about 1.1:1 to about 7:1,
iii. the amiodarone is dissolved while maintaining a solution temperature of about 15° C. to about 65° C. and a pH less than or equal to the pKa of amiodarone, to give a clear solution absent of gel,

b. preparing a second aqueous solution containing one or more components selected from pH adjusting agents, buffering agents, tonicity modifying agents, antioxidants, and additional sulfobutylether beta-cyclodextrin,

c. combining the two solutions and mixing the resulting solution to give a clear solution absent of gel,

d. optionally adding additional water and sulfobutylether beta-cyclodextrin as necessary to provide a final solution with an amiodarone concentration of 0.73 to 7.3 mM and a cyclodextrin:amiodarone mole ratio of greater than about 2.7:1 to about 7:1.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% absorptive loss of amiodarone from the solution in the container, when stored at room temperature for 6 months.

In certain embodiments, there is formation of no more than about 0.1% (w/w) total impurities in the solution in the container, when stored at room temperature for 6 months.

Another embodiment encompasses a method for making a ready to use pharmaceutical composition free of visible particulates comprising the steps of:

a. adding a volume of water for injection equivalent to 20% to 30% of the final solution volume to a suitable container and adjusting the temperature to 20-30° C.,

b. adding and dissolving sulfobutylether 7 beta-cyclodextrin, citric acid and sodium citrate such that the solution pH is in the range of about 3 to about 4,

c. adding and dissolving amiodarone hydrochloride in an amount sufficient to provide a cyclodextrin:amiodarone mole ratio in the final solution of about 3.1:1 to 3.2:1 to give a clear solution absent of gel,

d. adding additional water for injection to bring the solution volume to 70-80% of the final solution volume, and dissolving sufficient dextrose to provide a final solution osmolality of about 255 mOsm/kg to about 345 mOsm/kg,

e. adjusting the pH to 3.55 -3.65 with hydrochloric acid or sodium hydroxide,

f. adding additional water for injection to bring the solution to a final solution volume wherein the amiodarone concentration is 2 -3 mM and the citrate concentration ranges from about 2 to 3 mM,

g. sterile filtering the solution to substantially reduce microbial contamination, and

h. aseptically filling the solution into a pharmaceutically acceptable container.

In certain embodiments, the method further comprises adding one or more components selected from pH adjusting agents, buffering agents, antioxidants and tonicity modifying agents to the solution.

In certain embodiments, the final solution contains about 0.9 mM to about 5 mM citrate buffering agent.

In certain embodiments, the cyclodextrin:amiodarone mole ratio ranges from about 2.9:1 to about 5:1.

In certain embodiments, the pH during amiodarone dissolution ranges from about 3 to about 4.5.

In certain embodiments, the final solution sulfobutylether beta-cyclodextrin concentration ranges from about 13 mg/mL to about 40 mg/mL and the amiodarone concentration ranges from about 2.2 to 2.7 mM.

In certain embodiments, the surface tension of the final solution ranges from about 59 dynes/cm to about 75 dynes/cm.

In certain embodiments, the final solution has a pH of about 3 to about 4.

In certain embodiments, the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

In certain embodiments, the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

In certain embodiments, the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

In certain embodiments, there is no more than about 1% absorptive loss of amiodarone from the solution in the container, when stored at room temperature for 6 months.

In certain embodiments, there is formation of no more than about 0.1% (w/w) total impurities in the solution in the container, when stored at room temperature for 6 months.

In certain embodiments, the pharmaceutically acceptable container is a flexible bag comprised of one or more of ethylene vinyl acetate, polyolefin, polypropylene, polyethylene, polyvinylchloride, nylon, and/or combinations thereof.

In certain embodiments, the final solution volume comprises amiodarone at a concentration of about 2.5 mM to about 2.8 mM and the citrate concentration ranges from about 2 mM to about 3 mM.

### Compositions of the Invention

The invention encompasses compositions indicated for the treatment and prophylaxis of frequently recurring ventricular fibrillation and hemodynamically unstable ventricular tachycardia in patients refractory to other therapy.

The invention is based in part on the finding that the safety and efficacy of amiodarone formulations in bag formulations are unexpectedly improved by the removal of polysorbate 80 and benzyl alcohol.

The invention is also based in part on the finding that certain compositions containing amiodarone and a substituted cyclodextrin show reduced foaming when agitated during manufacturing and handling, thus improving the ability to manufacture and to inspect the solutions for particulate matter.

The compositions of the invention encompasses liquid formulations including amiodarone or a salt thereof that can be administered parenterally, for example, intravenously, to a subject in need thereof.

In one embodiment, the compositions of the invention encompass an aqueous liquid formulation including amiodarone or a salt thereof complexed with a substituted cyclodextrin in a bag suitable for injection to a subject. In certain embodiments, the substituted cyclodextrin is a sulfoalkyl ether cyclodextrin, a sulfoalkyl ether-alkyl ether cyclodextrin, a sulfoalkyl ether-hydroxyalkyl ether cyclodextrin or a hydroxyalkyl ether cyclodextrin.

In an illustrative embodiment, the invention encompasses an aqueous bag formulation for example for intravenous administration including amiodarone or a salt thereof, as an active ingredient, solubilized by complexation and/or non-covalent ionic bonding with sulfoalkyl ether cyclodextrin to a concentration range of about 0.1 mg/mL to about 5 mg/mL , in certain embodiments about 0.5 mg/mL to about 4 mg/mL, in other embodiments about 0.75 mg/mL to about 3 mg/mL, or in other embodiments about 1 mg/mL to about 2 mg/mL in aqueous solution. In certain embodiments the concentration is 1.5 mg/mL. In other embodiments, the concentration is 1.8 mg/mL. The formulation optionally includes one or more tonicity modifying agents; one or more buffering agents, one or more antioxidants, and one or more pH adjusting agents. In certain embodiments, the solution has a pH in the range of about 2 to about 6. In another embodiment, the molar ratio of sulfoalkyl ether cyclodextrin to amiodarone ranges from about 2.7:1 to 7:1 or in certain embodiments about 2.9:1 to 5:1 or about 3.2:1. In another embodiment, the formulation is sterilized and aseptically filled into a pharmaceutically acceptable container. In certain embodiments, the container is a flexible bag comprised of one or more of polyolefin, polyethylene, and polyvinylchloride.

In another embodiment, the invention encompasses an aqueous bag formulation for parenteral administration including amiodarone or a salt thereof, as an active ingredient, complexed with sulfoalkyl ether beta-cyclodextrin, wherein the amiodarone is at a concentration range of about 1.5 mg/ml in aqueous solution. The formulation also includes one or more tonicity modifying agents; one or more buffering agents, and one or more pH adjusting agents. In certain exemplary embodiments, the formulation requires no dilution before administration and has a pH of about 3.0 to about 5.0. In other embodiments, the molar ratio of SAE-CD to amiodarone is about 3:1 or about 3.2:1. In other embodiments, the formulation of the invention has been sterilized, for example, sterile filtered; and aseptically filled into a pharmaceutically acceptable container.

In another embodiment, the invention encompasses an aqueous bag formulation for parenteral administration including amiodarone or a salt thereof, as an active ingredient, complexed with sulfoalkyl ether beta-cyclodextrin, wherein the amiodarone is at a concentration range of about 1.8 mg/ml in aqueous solution. The formulation also includes one or more tonicity modifying agents; one or more buffering agents, and one or more pH adjusting agents. In certain exemplary embodiments, the formulation requires no dilution before administration and has a pH of about 3.0 to about 5.0. In other embodiments, the molar ratio of SAE-CD to amiodarone is about 3:1 or about 3.2:1. In other embodiments, the formulation of the invention has been sterilized, for example, sterile filtered; and aseptically filled into a pharmaceutically acceptable container.

According to the invention, there is provided a parenteral (e.g., intravenous) liquid formulation containing as an active ingredient amiodarone hydrochloride, which is solubilized by complexation and/or non-covalent ionic bond with SAE-CD in a citric acid/sodium citrate buffer.

In other embodiments, the invention encompasses compositions containing a substituted cyclodextrin comprising a mixture of two or more different degrees of substitution. The resulting substituted cyclodextrin will have an average degree of substitution. The invention also provides compositions containing a substituted cyclodextrin having a single degree of substitution such as four or six, wherein each cyclodextrin molecule has four or six substituents respectively.

In other embodiments, the invention encompasses compositions containing a mixture of substituted cyclodextrins, each containing a different substituent, for example a mixture of SAE-CD and HAE-CD. The invention also provides compositions containing a cyclodextrin derivative wherein more than one substituent type is present on a single cyclodextrin ring such as SAE-AE-CD and SAE-HAE-CD.

In certain of the embodiments of the invention unreacted cyclodextrin has been substantially removed, with the remaining impurities (i.e., <5 wt. % of composition) being inconsequential to the performance of the cyclodextrin derivative-containing composition.

Exemplary SAE-CD derivatives include SBE4-β-CD, SBE7-β-CD, SBE11-β-CD, and SBE4-γ-CD which correspond to SAE-CD derivatives of the formula I wherein n=5, 5, 5 and 6; m is 4; and there are on average 4, 7, 11 and 4 sulfoalkyl ether substituents present, respectively.

Although not necessary, the formulation of the present invention may include an antioxidant, buffering agent, a pH adjusting agent, acidifying agent, alkalizing agent, complexation enhancing agent, solvent, electrolyte, salt, dextrose, water, glucose, tonicity modifier, antifoaming agent, or a combination thereof.

The formulation of the invention also includes water. Specific embodiments of the invention include pyrogen-free, sterile water as liquid carrier. The water can comprise other components described herein. Water suitable for injection is suitable for use in the liquid formulation of the invention.

The formulation of the invention can also include biological salt(s), sodium chloride, potassium chloride, or other electrolyte(s).

An antioxidant may be but need not be added to the formulation of the invention. Preferred antioxidants include EDTA, sodium metabisulfite and pentetate, for example.

The chemical stability of the liquid formulations of the invention can be enhanced by: adding an antioxidant, adjusting the pH of the liquid carrier, and/or eliminating or minimizing the presence of oxygen in the formulation.

Tables 1 and 2 describe illustrative embodiments of the formulations of the invention.

TABLE 1

| | 200 mL (Bag for IV) | | 100 mL (Bag for IV) | |
|---|---|---|---|---|
| | 360 mg/200 mL | | 150 mg/100 mL | |
| | SAE-CD | HAE-CD | SAE-CD | HAE-CD |
| | formu- | formu- | formu- | formu- |
| | lation | lation | lation | lation |
| Amiodarone | 1.8 mg/mL | 1.8 mg/mL | 1.5 mg/mL | 1.5 mg/mL |
| HCl | | | | |
| Sulfobutyl | 18 mg/mL | 0 | 15 mg/mL | 0 |
| ether beta | | | | |
| cyclodextrin | | | | |
| 2-hydroxy- | 0 | 30 mg/mL | 0 | 25 mg/mL |
| propyl beta | | | | |
| cyclo- | | | | |
| dextrin | | | | |
| Citric Acid | 0.4 mg/mL | 0.4 mg/mL | 0.4 mg/mL | 0.4 mg/mL |
| Anhydrous, | | | | |
| USP | | | | |
| Sodium Citrate | 0.2 mg/mL | 0.2 mg/mL | 0.2 mg/mL | 0.2 mg/mL |
| Dihydrate, | | | | |
| USP | | | | |
| Dextrose | 41 mg/mL | 45 mg/mL | 44 mg/mL | 45 mg/mL |
| Anhydrous, | | | | |
| USP | | | | |
| Hydrochloric | As required | As required | As required | As required |
| Acid, NF or | to achieve a | to achieve a | to achieve a | to achieve a |
| Sodium | pH about 3.6 | pH about 3.6 | pH about 3.6 | pH about 3.6 |
| Hydroxide, NF | | | | |

TABLE 2

| | 200 mL (Bag for IV) | | 100 mL (Bag for IV) | |
|---|---|---|---|---|
| | 360 mg/200 mL | | 150 mg/1100 mL | |
| | SAE-CD | HAE-CD | SAE-CD | HAE-CD |
| | formulation | formulation | formulation | formulation |
| Amiodarone | 1.8 mg/mL | 1.8 mg/mL | 1.5 mg/mL | 1.5 mg/mL |
| HCl | | | | |
| Sulfobutyl | 18 mg/mL | 0 | 15 mg/mL | 0 |
| ether beta | | | | |
| cyclodextrin | | | | |
| 2-hydroxy- | 0 | 30 mg/mL | 0 | 25 mg/mL |
| propyl beta | | | | |
| cyclo- | | | | |
| dextrin | | | | |
| Citric Acid | 0.4 mg/mL | 0.4 mg/mL | 0.4 mg/mL | 0.4 mg/mL |
| Anhydrous, | | | | |
| USP | | | | |
| Sodium Citrate | 0.2 mg/mL | 0.2 mg/mL | 0.2 mg/mL | 0.2 mg/mL |
| Dihydrate, | | | | |
| USP | | | | |
| Sodium | 9 mg/mL | 9 mg/mL | 9 mg/mL | 9 mg/mL |
| Chloride, | | | | |
| USP | | | | |
| Hydrochloric | As required | As required | As required | As required |
| Acid, NF or | to achieve a | to achieve a | to achieve a | to achieve a |
| Sodium | pH about 3.6 | pH about 3.6 | pH about 3.6 | pH about 3.6 |
| Hydroxide, NF | | | | |

In certain embodiments, the mole ratio of the SAE-CD to amiodarone HCl is in the range of about 2.7:1 to about 7:1, about 2.9:1 to about 5:1, or about 3:1 to about 3.5:1.

In other embodiments, the mole ratio of the HAE-CD to amiodarone HCl ranges from about 5:1 to about 14:1, in other embodiments about 7:1 to about 11:1 or in other embodiments about 8:1.

In other embodiments, the mole ratio of the SAE-HAE-CD to amiodarone HCl ranges from about 2.5:1 to about 7:1.

In other embodiments, the mole ratio of the SAE-AE-CD to amiodarone HCl ranges from about 2.5:1 to about 7:1.

In other embodiments, the amount of citric acid and sodium citrate can be varied to achieve buffer concentrations of 0.9-5.0 mM, for example 2.5 mM. In other embodiments, dextrose is used to maintain osmolality in the range of about 255 to 345 mOsm/kg. In other embodiments, sodium chloride is used to maintain osmolality in the range of about 255 to 345 mOsm/kg. Sodium hydroxide or hydrochloric acid can be used in certain embodiments to adjust pH, for example to a pH of about 3 to about 4, for example to a pH of about 3.3 to about 3.9, for example about 3.6.

In certain embodiments, the formulation of the invention has a surface tension in the range of about 59 to 75 dynes/cm. In other embodiments, the formulation of the invention shows less foaming upon agitation than formulations not containing a substituted cyclodextrin.

In certain embodiments, the formulations of the invention are physically and chemically stable when stored in pharmaceutically acceptable plastic or glass containers at room temperature. In other embodiments, the formulations of the invention form no more than about 0.1% (w/w) total impurities in the solution in the container, when stored at room temperature for 6 months. In other embodiments, the formulations of the invention have no or low amounts of adsorption of the amiodarone to the container. In certain embodiments, the adsorptive loss of amiodarone to the container is no more than 1% (w/w) when stored at room temperature for 6 months.

The formulations of the invention avoid the adverse effects associated with ingredients in the currently marketed amiodarone compositions. For example, the formulations of the invention do not include benzyl alcohol or polysorbate 80. Accordingly, the formulations of the invention can be administered to populations that are at risk of adverse effects associated with current amiodarone compositions. The invention therefore encompasses methods of treating or preventing disorders in patients that were not able to receive current amiodarone compositions. In one embodiment, the invention is administered to neonates and overcomes the fatal gasping associated with currently marketed formulations of amiodarone.

### Methods of Making the Compositions of the Invention

The invention is based in part on the finding that certain compositions allow preparation of the formulations at elevated temperatures without formation of a gel, thus reducing the time required for dissolution of the ingredients. This can be accomplished by several procedures including using a sufficiently high cyclodextrin to amiodarone mole ratio during dissolution of the amiodarone, or using lower mole ratios but conducting the amiodarone dissolution in a smaller initial volume whereby the concentration of all components in that solution are increased relative to the final formulation. Alternatively, a broad range of cyclodextrin to amiodarone mole ratios can be used while still avoiding the formation of gel provided the temperature during dissolution of the amiodarone is maintained in a lower range.

The invention is also based in part on the finding that certain compositions prevent the formation of visible particulate matter when the formulations are stored in pharmaceutically acceptable containers. This can be accomplished primarily by preparing the formulation with a sufficiently high cyclodextrin to amiodarone mole ratio in the final formulation. The mole ratio required for preventing the formation of visible particulate matter is also sufficient for avoiding the formation of gel during dissolution of the amiodarone, but certain mole ratios that prevent gel formation do not prevent particulate formation upon storage.

The invention is also based in part on the finding that certain compositions prevent or minimize the formation of foam during manufacture or upon agitation, for example during a process for visual inspection for visible particulates. Increasing the cyclodextrin to amiodarone mole ratio will decrease the potential for formation of foam during agitation.

The liquid formulations of the invention can be prepared by numerous different methods.

In one embodiment, the methods encompass a one-tank procedure wherein a solution comprising a substituted cyclodextrin and amiodarone or a salt thereof and further including additional agents, for example, buffers and tonicity agents is prepared. The solution is mixed to form the liquid formulation. The solution can independently comprise other excipients and agents described herein. In this procedure, conditions including temperature, pH, and stirring can be controlled to optimize the procedure.

In one embodiment, the methods encompass a procedure wherein an aqueous solution is prepared comprising amiodarone or a pharmaceutically acceptable salt thereof and a sulfoalkyl ether cyclodextrin in an amount providing a cyclodextrin:amiodarone mole ratio of greater than about 2.7:1 to 7:1 and the amiodarone is dissolved at a pH which is less than the pKa of amiodarone, to give a solution absent, for example, visibly absent of gel. The solution is further diluted with water as necessary to provide a formulation containing about 0.7 to 7 mM amiodarone. In certain embodiments the temperature of the solution during dissolution of the amiodarone is in the range of about 15° C. to 65° C. In certain embodiments, the solution has a pH in the range of about 2 to 6 or about 3 to 4.5. In certain embodiments the cyclodextrin:amiodarone mole ratio in the range of about 2.9:1 to 5:1. In certain embodiments, the formulation is sterile filtered and filled into a pharmaceutically acceptable container. In other embodiments, the solution does not form visible particulates upon storage.

In an illustrative embodiment, the methods encompass a procedure wherein an aqueous solution is prepared comprising amiodarone or a pharmaceutically acceptable salt thereof and sulfobutyl ether beta-cyclodextrin in an amount providing a cyclodextrin:amiodarone mole ratio of greater than about 2.7:1 to 7:1 or in certain embodiments about 2.9:1 to 5:1 or about 3.2:1 and the amiodarone is dissolved at a pH which is less than the pKa of amiodarone, to give a solution absent of visible gel. The solution is further diluted with water as necessary to provide a formulation containing about 0.7 to 7 mM amiodarone. In certain embodiments the formulation optionally includes one or more tonicity modifying agents; one or more buffering agents, one or more antioxidants, and one or more pH adjusting agents. In certain embodiments, the amiodarone is dissolved at a temperature of about 15° C. to 65° C. or about 15° C. to 57° C. In other embodiments, the amiodarone is dissolved at a pH of about 3 to 4.5. In another embodiment, the formulation is sterilized and aseptically filled into a pharmaceutically acceptable container. In certain embodiments, the container is a flexible bag comprised of one or more of polyolefin, nylon, polyethylene, polyvinylidene chloride, and polyvinylchloride.

In another embodiment, the methods encompass a procedure wherein an aqueous solution is prepared comprising amiodarone or a pharmaceutically acceptable salt thereof and a sulfoalkyl ether cyclodextrin in an amount providing a cyclodextrin:amiodarone mole ratio of about 1.1:1 to 7:1 and the amiodarone is dissolved at a pH which is less than the pKa of amiodarone and a temperature of about 15° C. to less than about 40° C. to give a solution absent, for example, visibly absent of gel. Additional sulfoalkyl ether cyclodextrin is then added as needed to provide a solution with a cyclodextrin:amiodarone mole ratio of greater than about 2.7:1 to 7:1. The solution is further diluted with water as necessary to provide a formulation containing about 0.7 to 7 mM amiodarone. In certain embodiments the temperature of the solution during dissolution of the amiodarone is in the range of about 15° C. to 35° C. In certain embodiments, the solution has a pH in the range of about 2 to 6 or about 3 to 4.5. In certain embodiments, the formulation is sterile filtered and filled into a pharmaceutically acceptable container. In other embodiments, the solution does not form visible particulates upon storage.

In another embodiment, the methods encompass a procedure wherein an aqueous solution is prepared comprising amiodarone or a pharmaceutically acceptable salt thereof and a sulfoalkyl ether cyclodextrin in an amount providing a cyclodextrin:amiodarone mole ratio of about 1.5:1 to 7:1 and the amiodarone is dissolved at a pH which is less than the pKa of amiodarone and a temperature of about 15° C. to about 65° C. to give a solution absent, for example, visibly absent of gel. Additional sulfoalkyl ether cyclodextrin is then added as needed to provide a solution with a cyclodextrin:amiodarone mole ratio of greater than about 2.7:1 to 7:1. The solution is further diluted with water as necessary to provide a formulation containing about 0.7 to 7 mM amiodarone. In certain embodiments the temperature of the solution during dissolution of the amiodarone is in the range of about 15° C. to 57° C. In certain embodiments, the solution has a pH in the range of about 2 to 6 or about 3 to 4.5. In certain embodiments, the final solution has a cyclodextrin:amiodarone mole ratio of about 2.9:1 to 3.5:1. In certain embodiments, the formulation is sterile filtered and filled into a pharmaceutically acceptable container. In other embodiments, the solution does not form visible particulates upon storage.

In another embodiment, the methods encompass a procedure wherein a concentrated aqueous solution comprising amiodarone or a pharmaceutically acceptable salt thereof and a sulfoalkyl ether cyclodextrin is prepared in a volume representing from about 1% to less than about 17% of the volume of the final solution, the cyclodextrin:amiodarone mole ratio is in the range of about 1.1:1 to 7:1 and the amiodarone is dissolved at a pH which is less than the pKa of amiodarone and a temperature of about 15° C. to about 65° C. to give a solution absent, for example, visibly absent of gel. Additional water and sulfoalkyl ether cyclodextrin is then added as needed to provide a final solution containing about 0.7 to 7 mM amiodarone and a cyclodextrin:amiodarone mole ratio of greater than about 2.7:1 to 7:1. In certain embodiments the temperature of the solution during dissolution of the amiodarone is in the range of about 15° C. to 57° C. In certain embodiments, the solution has a pH in the range of about 2 to 6 or about 3 to 4.5. In certain embodiments, the final solution has a cyclodextrin:amiodarone mole ratio of about 2.9:1 to about 3.5:1. In certain embodiments, the formulation is sterile filtered and filled into a pharmaceutically acceptable container. In other embodiments, the solution does not form visible particulates upon storage.

In various embodiments, the methods for preparing the liquid formulation can encompass addition of one or more tonicity modifying agents; one or more buffering agents, one or more antioxidants, and one or more pH adjusting agents to the solution. In certain embodiments, dextrose is added to the solution to provide an osmolality in the final solution in the range of about 255 to 345 mOsm/kg. In certain other embodiments, sodium chloride is added to the solution to provide an osmolality in the final solution in the range of about 255 to 345 mOsm/kg.

In one embodiment, the methods encompass a two-tank procedure wherein a first aqueous solution comprising a substituted cyclodextrin and amiodarone or a salt thereof is prepared. A second aqueous solution including one or more additional agents, for example, buffers and tonicity agents is prepared. The first and second solutions are mixed to form the liquid formulation final solution. The first and second solutions can independently comprise other excipients and agents described herein. In this procedure, conditions including temperature, pH, and stirring can be controlled to optimize the procedure.

In one embodiment, the first aqueous solution comprises sulfoalkyl ether cyclodextrin and amiodarone or a pharmaceutically acceptable salt thereof in a cyclodextrin:amiodarone mole ratio of about 1.1:1 to 7:1, the volume of the first solution represents from about 1% to less than about 17% of the volume of the final solution, and the amiodarone is dissolved at a temperature of about 15° C. to 65° C. and a pH less than or equal to the pKa of amiodarone. Additional cyclodextrin is added as necessary to the first solution and/or the second solution such that the cyclodextrin:amiodarone mole ratio in the final solution is in the range of greater than about 2.7:1 to 7:1.

In various embodiments, the methods for preparing the liquid formulation can encompass in the case of a two-tank procedure the steps of heating either the first solution or heating the second solution, or heating a combination thereof of any solutions described in the above methods followed by the step of cooling the respectively heated solution. In the case of a one-tank procedure, the steps of heating the solution in the above methods is followed by the step of cooling the respectively heated solution.

The method for preparing the liquid formulation also includes the step of adjusting the pH of either the first solution or adjusting the pH of the second solution or adjusting the pH of a combination of either solution.

### VI. EXAMPLES

In view of the above description and the examples below, one of ordinary skill in the art will be able to practice the invention as claimed without undue experimentation. The foregoing will be better understood with reference to the following examples that detail certain procedures for the preparation of formulations according to the present invention. All references made to these examples are for the purposes of illustration and not limitation. The following examples should not be considered exhaustive or exclusive, but merely illustrative of only a few of the many embodiments contemplated by the invention.

### Example 1

### Solubilization of Amiodarone HCl with a Sulfobutylether beta-Cyclodextrin

A volume of deionized water was added to a compounding vessel and brought to a desired temperature which was maintained throughout the study. The cyclodextrin and citric acid, when present, were added and dissolved with stirring provided by an overhead mixer. The amiodarone HCl was slowly added with continued stirring and the vessel contents visually observed for the presence of gel. Stirring continued until the amiodarone was dissolved. Batch parameters and results are in the table below.

| Component or | Batch | | | | | | |
|---|---|---|---|---|---|---|---|
| Condition | 1a | 1b | 1c | 1d | 1e | 1f | 1g |
| Amiodarone | 0.9 | 0.9 | 0.9 | 0.9 | 0.9 | 0.9 | 0.9 |
| hydrochloride (g) | | | | | | | |
| Sulfobutylether | 4.05 | 4.05 | 4.05 | 4.05 | 4.05 | 4.05 | 4.05 |
| betacyclo- | | | | | | | |
| dextrin* (g) | | | | | | | |
| Citric acid | 0 | 0 | 0 | 0.37 | 0.07 | 0.37 | 0.07 |
| monohydrate (g) | | | | | | | |
| Water (L) | 0.5 | 0.15 | 0.5 | 0.15 | 0.15 | 0.15 | 0.15 |
| Temperature | 57 | 57 | 25 | 40 | 40 | 35 | 35 |
| (° C.) | | | | | | | |
| Gel formation | Yes | Yes | No | Yes | Yes | No | No |

*Captisol ®

Formulations containing cyclodextrin:amiodarone mole ratios of about 1.4:1 form gels when prepared at temperatures of about 40° C. to 57° C.

### Example 2

### Solubilization of Amiodarone HCl in the Presence of Dextrose

Formulations were prepared in deionized water according to the following formula:

| Component | Content (g/L) |
|---|---|
| Amiodarone hydrochloride | 1.8 |
| Sulfobutylether beta- | 8.1 |
| cyclodextrin (“SBECD”)* | |
| Dextrose (anhydrous) | 45.5 |
| Citric acid (anhydrous) | 0.68 for 5 mM buffer |
| Citric acid (monohydrate) | 0.36 for 2.5 mM buffer |
| | 0.14 for 0.9 mM buffer |
| Sodium citrate (dihydrate) | 0.43 for 5 mM buffer |
| | 0.18 for 2.5 mM buffer |
| | 0.08 for 0.9 mM buffer |
| Deionized water | QS to final volume |

*Captisol ®

An initial volume of deionized water was added to a formulation vessel and brought to a set temperature. The dextrose, citric acid, SBECD and sodium citrate were each added and dissolved with mixing. The pH of the solution was measured, and the amiodarone HCl was slowly added with vigorous mixing. The solutions were observed for the presence of gel formation. If no gel formation was observed, the solutions were brought to room temperature as needed and brought to their final volume with deionized water. Formulation parameters and results are indicated in the table below.

| | | | Initial | | | |
|---|---|---|---|---|---|---|
| | | Final | Volume | | pH before | |
| | Buffer | Volume | (% of | Temp. | amiodarone | Gel |
| Batch | (mM) | (L) | Final) | (° C.) | addition | Formation |
| 2a | 5 | 2 | 90 | 25 | 3.55 | No |
| 2b | 5 | 2 | 90 | 32.5 | 3.52 | No |
| 2c | 5 | 2 | 90 | 57 | 3.64 | Yes |
| 2d | 5 | 75 | 30 | 25 | 3.53 | No |
| 2e | 5 | 2.6 | 30 | 35 | 3.48 | No |
| 2f | 5 | 2.6 | 30 | 57 | not measured | Yes |
| 2g | 5 | 75 | 20 | 25 | 3.6 | No |
| 2h | 2.5 | 1500 | 20 | 15 | 3.58 | No |
| 2i | 2.5 | 175 | 20 | 25 | 3.6 | No |
| 2j | 2.5 | 1 | 20 | 22 | not measured | No |
| 2k | 2.5 | 1 | 20 | 30 | not measured | No |
| 2m | 2.5 | 1 | 20 | 45 | not measured | Yes |
| 2n | 2.5 | 1 | 20 | 60 | not measured | Yes |
| 2p | 2.5 | 1.14 | 17.5 | 45 | not measured | Yes |
| 2q | 2.5 | 1.33 | 15 | 45 | not measured | No |
| 2r | 2.5 | 1.6 | 12.5 | 45 | not measured | No |
| 2s | 2.5 | 1 | 10 | 25 | not measured | No |
| 2t | 2.5 | 1 | 10 | 45 | not measured | No |
| 2u | 0.9 | 3 | 90 | 25 | 3.83 | No |
| 2v | 0.9 | 75 | 40 | 25 | 3.45 | No |
| 2w | 0.9 | 0.1 | 40 | 42 | 3.29 | Yes |
| 2x | 0.9 | 4 | 40 | 54 | 3.62 | Yes |
| 2y | 0.9 | 75 | 26.6 | 25 | 3.47 | No |

Formulations containing cyclodextrin:amiodarone mole ratios of about 1.4:1 do not form gels at dissolution temperatures of 45° C. or higher when prepared at initial volumes less than about 17.5% (corresponding to SBECD concentrations of greater than about 46 mg/mL).

### Example 3

### Solubilization of Amiodarone HCl with Different Order of Addition

Formulations were prepared as in Example 2 except the sodium citrate was added after the amiodarone was dissolved. The formulation parameters and results are indicated in the table below.

| | | | Initial | | | |
|---|---|---|---|---|---|---|
| | | Final | Volume | | pH before | |
| | Buffer | Volume | (% of | Temp. | amiodarone | Gel |
| Batch | (mM) | (L) | Final) | (° C.) | addition | Formation |
| 3a | 5 | 2 | 90 | 30 | 3.5 | No |
| 3b | 5 | 2 | 90 | 57 | 3.53 | Yes |
| 3c | 5 | 75 | 20 | 34 | 3.54 | No |
| 3d | 0.9 | 75 | 20 | 30 | 3.71 | No |
| 3e | 0.9 | 75 | 20 | 34 | 3.77 | No |
| 3f | 0.5 | 200 | 80 | 57 | 3.5 | Yes |

### Example 4

### Solubilization of Amiodarone HCl with Different Mole Ratios of SBECD

Formulations were prepared in deionized water according to the following formula:

| Component | Content (g/L) |
|---|---|
| Amiodarone hydrochloride | 1.8 |
| Sulfobutylether 7 beta-cyclodextrin* | 8.1-20 |
| (“SBECD”) | |
| Dextrose (anhydrous) | 45.5 |
| Citric acid (monohydrate) | 0.36 |
| Sodium citrate (dihydrate) | 0.18 |
| Deionized water | QS to 1 L |

*Captisol ®

An initial 200 mL volume of deionized water was added to the vessels of a Varian VanKel dissolution apparatus and brought to a set temperature. The dextrose, citric acid, SBECD and sodium citrate were each added and dissolved with overhead mixing at 250 rpm. The amiodarone HCl was slowly added with continued mixing. The solutions were observed for the presence of gel formation. If no gel formation was observed, the solutions were cooled to room temperature as needed and brought to a final volume of 1 L with deionized water. Formulation parameters and results are indicated in the table below.

| | SBECD Content in | | | |
|---|---|---|---|---|
| | Final Volume | Buffer | Temp. | Gel |
| Batch | (g/L) | (mM) | (° C.) | Formation |
| 4a | 8.1 | 2.5 | 25 | No |
| 4b | 10 | 2.5 | 25 | No |
| 4c | 15 | 2.5 | 25 | No |
| 4d | 20 | 2.5 | 25 | No |
| 4e | 8.1 | 2.5 | 45 | Yes |
| 4f | 8.6 | 2.5 | 45 | No |
| 4g | 9.3 | 2.5 | 45 | No |
| 4h | 9.8 | 2.5 | 45 | No |
| 4i | 10 | 2.5 | 45 | No |
| 4j | 15 | 2.5 | 45 | No |
| 4k | 20 | 2.5 | 45 | No |

Increasing the SAE-CD concentration during dissolution by increasing the SAE-CD:Amiodarone mole ratio to a value of about 1.5 or above at a fixed initial volume allows for increasing the temperature during dissolution without gel formation.

### Example 5

### Effects of Cyclodextrin Content on Dissolution Time and Surface Tension

Formulations were prepared with varying amounts of SBECD in deionized water according to the following formula:

| Component | Content (g/L) |
|---|---|
| Amiodarone hydrochloride | 1.8 |
| Sulfobutylether 7 beta-cyclodextrin* | 8.1-20 |
| (“SBECD”) | |
| Dextrose (anhydrous) | 45.5 |
| Citric acid (anhydrous) | 0.36 |
| Sodium citrate (Dihydrate) | 0.18 |
| Deionized water | QS to 1 L |

*Captisol ®

An initial volume of 200 mL deionized water was added to a vessel in a Varian VanKel dissolution apparatus and brought to 25° C. The citric acid and SBECD were added and dissolved with stirring provided by the dissolution paddles rotating at 250 rpm. The amiodarone HCl was slowly added with continued stirring and the time for visually complete dissolution of the amiodarone was noted. The pH of the solution was then measured, the solution brought to 800 mL with water, and the sodium citrate and dextrose added and dissolved with stirring. The solutions were brought to final volume of 1 L with water and the pH and surface tension measured with a Fisher Scientific, Model 21 Tensiomat. The solutions were observed throughout the process for the presence of gel formation and none was noted. Placebo solutions were also prepared (no amiodarone addition) and the surface tension was measured for those solutions. Formulation parameters and results are indicated in the table below.

| | | | pH after | | Time for | Batch surface | Placebo surface |
|---|---|---|---|---|---|---|---|
| | SBECD | Buffer | amiodarone | pH at | dissolution | tension | tension |
| Batch | (g/L) | (mM) | addition | final volume | (min) | (dynes/cm) | (dynes/cm) |
| 5a | 8.1 | 2.5 | 2.77 | 3.85 | ~150 | 53.1 | 74 |
| 5b | 10 | 2.5 | 2.77 | 3.84 | ~60 | 57.6 | 74.6 |
| 5c | 13 | 2.5 | 2.78 | 3.85 | <30 | 60.1 | 74.8 |
| 5d | 15 | 2.5 | 2.78 | 3.83 | <30 | 62 | 74.7 |
| 5e | 20 | 2.5 | 2.80 | 3.85 | <30 | 65 | 74.4 |

Increasing the cyclodextrin content led to shorter dissolution times and increased surface tension. Less foaming was observed as the surface tension increased.

### Example 6

### Formulations Prepared with Sodium Chloride

Formulations were prepared in deionized water according to the following formula:

| Component | Content (g/L) |
|---|---|
| Amiodarone Hydrochloride | 1.8 |
| Sulfobutylether beta- | 8.1 |
| cyclodextrin* (“SBECD”) | |
| Sodium Chloride | 9 |
| Citric acid (anhydrous) | 0.68 for 5 mM buffer |
| Citric acid (monohydrate) | 0.14 for 0.9 mM buffer |
| | 0.43 for 5 mM buffer |
| Sodium Citrate (Dihydrate) | 0.08 for 0.9 mM buffer |
| Deionized water | QS to final volume |

*Captisol ®

An initial volume of deionized water was added to a formulation vessel and brought to a set temperature. The citric acid, sodium citrate and SBECD were each added and dissolved with mixing. The pH of the solution was measured, and then the amiodarone HCl was slowly added with vigorous mixing. The sodium chloride was then added and dissolved with mixing. The solutions were observed for the presence of gel formation. If no gel formation was observed, the solutions were brought to their final volume with deionized water. Formulation parameters and results are indicated in the table below.

| | | | Initial | | | |
|---|---|---|---|---|---|---|
| | | Final | Volume | | pH before | |
| | Buffer | Volume | (% of | Temp. | amiodarone | Gel |
| Batch | (mM) | (L) | Final) | (° C.) | addition | Formation |
| 6a | 5 | 2 | 90 | 57 | 3.67 | Yes |
| 6b | 0.9 | 40 | 90 | 57 | 3.8 | Yes |

### Example 7

### Formulations Prepared with Sodium Chloride and Varying Cyclodextrin Content

Formulations were prepared in deionized water according to the following formula:

| | Content of final solution (g/L) | | | | | |
|---|---|---|---|---|---|---|
| | Batch | Batch | Batch | Batch | Batch | Batch |
| Component | 7a | 7b | 7c | 7d | 7e | 7f |
| Amiodarone | 1.8 | 1.8 | 1.8 | 1.8 | 1.8 | 1.8 |
| hydrochloride | | | | | | |
| Sulfobutylether | 8.1 | 13 | 15 | 20 | 20 | 20 |
| β-cyclodextrin* | | | | | | |
| Citric acid | 0.36 | 0.36 | 0.36 | 0.36 | 0.36 | 0.36 |
| (anhydrous) | | | | | | |
| Sodium citrate | 0.18 | 0.18 | 0.18 | 0.18 | 0.18 | 0.18 |
| (dihydrate) | | | | | | |
| Sodium chloride | 9 | 9 | 9 | 9 | 9 | 9 |
| Deionized water | QS | QS | QS | QS | QS | QS |
| Final pH | 3.7-3.8 | 3.7-3.8 | 3.7-3.8 | 3.7-3.8 | 3.6 | 4.5 |

*Captisol ®

An initial volume of 200 mL deionized water was added to a formulation vessel and brought to 25° C. The citric acid, sodium citrate and cyclodextrin were each added and dissolved with mixing provided with an overhead mixer. The amiodarone HCl was slowly added and dissolved with continued mixing to give a clear solution. The solutions were observed for the presence of gel formation and none was observed. The pH was measured at 3.7-3.8 for all batches. The sodium chloride was added, mixing was continued for one hour, after which the solutions were brought to 1 L with water. The pH of batches 7e and 7f was adjusted to 3.6 and 4.5 respectively with sodium hydroxide or hydrochloric acid. Mixing continued for 24 hours. The solutions were observed for the presence of any precipitate or incomplete dissolution at various times after the sodium chloride addition. Observations are presented in the table below.

Particulate matter observed after sodium chloride addition

| Batch | 1 hr | 5 hr | 24 hr |
|---|---|---|---|
| 7a | Yes | Yes | Yes |
| 7b | Yes | Yes | Yes |
| 7c | Yes | Yes | No |
| 7d | No | No | No |
| 7e | No | No | No |
| 7f | No | No | No |

### Example 8

### Formulations Prepared with Sodium Chloride with Alternate Order of Addition at 45° C.

Formulations were prepared in deionized water according to the following formula:

| | Content of final solution (g/L) | | |
|---|---|---|---|
| Component | Batch 8a | Batch 8b | Batch 8c |
| Amiodarone hydrochloride | 1.8 | 1.8 | 1.8 |
| Sulfobutylether beta-cyclodextrin* | 13 | 15 | 20 |
| Citric acid (anhydrous) | 0.36 | 0.36 | 0.36 |
| Sodium citrate (dihydrate) | 0.18 | 0.18 | 0.18 |
| Sodium chloride | 9 | 9 | 9 |
| Deionized water | QS | QS | QS |
| Final pH | 3.7-3.8 | 3.7-3.8 | 3.7-3.8 |

*Captisol ®

An initial volume of 200 mL deionized water was added to a formulation vessel and brought to 45° C. The citric acid, sodium citrate, cyclodextrin and sodium chloride were each added and dissolved with mixing provided with an overhead mixer. The amiodarone HCl was slowly added and dissolved with continued mixing. The solutions were observed for the presence of gel formation. If none was observed, the solutions were brought to a final volume of 1 L with water and observed for the presence of particulate matter. The surface tension and osmolality (Advanced Micro Osmometer Model 3300) of each final solution was measured. Observations are presented in the table below.

| | Gel | Particulate matter | Osmolality | Surface tension |
|---|---|---|---|---|
| Batch | observed | observed | (mOsm/kg) | (dynes/cm) |
| 8a | No | No | 327 | 58.2 |
| 8b | No | No | 334 | 59.7 |
| 8c | No | No | 346 | 60.2 |

### Example 9

### Visual Inspection of 1.5 mg/mL Amiodarone HCl Formulations with Various Cyclodextrin Contents

Formulations were prepared in deionized water according to the following formula:

| | Batch | Batch | Batch | Batch | Batch | Batch |
|---|---|---|---|---|---|---|
| | 9a | 9b | 9c | 9d | 9e | 9f |
| Amiodarone | 1.5 | 1.5 | 1.5 | 1.5 | 1.5 | 1.5 |
| hydrochloride | | | | | | |
| Sulfobutylether | 6.75 | 10.0 | 11.7 | 12.8 | 14.2 | 15 |
| β-cyclodextrin* | | | | | | |
| Dextrose anhydrous | 45.5 | 44.2 | 43.5 | 43.0 | 42.4 | 42.1 |
| USP | | | | | | |
| Citric acid anhydrous, | 0.36 | 0.36 | 0.36 | 0.36 | 0.36 | 0.36 |
| USP | | | | | | |
| Sodium citrate | 0.18 | 0.18 | 0.18 | 0.18 | 0.18 | 0.18 |
| dihydrate USP | | | | | | |
| Water for injection | QS | QS | QS | QS | QS | QS |
| (“WFI”) | | | | | | |
| Final batch volume (L) | 1500 | 75 | 75 | 75 | 75 | 1800 |

*Captisol ®

A clean stainless steel mix tank with overhead stirring was filled to approximately to 20% of final batch volume with Water for Injection (“WFI”) and held at the temperature range of 20° C.-30° C. Citric acid, Captisol, and sodium citrate dihydrate were added to the mix tank in that order, and mixed for no less than (“NLT”) 5 minutes after each component was added to the mix tank. Amiodarone HCl was added to the mix tank over 10 minutes and mixed for 1 hour using an in-line high-shear mixer after completing the addition of amiodarone HCl to the mix tank. The tank was brought to 80% of final batch volume with WFI and dextrose was added to the mix tank and mixed for NLT 5 minutes. The tank was brought to 90% of final batch volume with WFI, mixed for NLT 5 minutes and the pH measured. The tank was brought to the final batch volume with WFI, mixed for NLT 5 minutes and the pH measured. The solution was sterile filtered and filled into 100 mL Galaxy® plastic containers. Filled containers were evaluated for the presence of visible particulates.

No gel formation was observed and foaming during and after amiodarone addition was reduced and dissipated more quickly as the cyclodextrin concentration increased. The results of the pH, assay and visual inspection are given in the table below.

| | | | | Product |
|---|---|---|---|---|
| | Cyclodextrin | Cyclodextrin: | pH at | Related |
| | Concentration | Amiodarone | final | Particulates |
| Batch | (g/L) | mole ratio | volume | Present |
| 9a | 6.7 | 1.4:1 | 3.58 | Yes |
| 9b | 10 | 2.1:1 | 3.58 | Yes |
| 9c | 11.7 | 2.4:1 | 3.60 | No |
| 9d | 12.8 | 2.7:1 | 3.59 | Yes |
| 9e | 14.2 | 3:1 | 3.60 | No |
| 9f | 15 | 3.2:1 | 3.62 | No |

### Example 10

### Visual Inspection of 1.8 mg/mL Amiodarone HCl Formulations with Various Cyclodextrin Contents

Formulations were prepared in deionized water according to the following formula:

Content (g/L)

| | Batch | Batch | Batch | Batch |
|---|---|---|---|---|
| | 10a | 10b | 10c | 10d |
| Amiodarone | 1.8 | 1.8 | 1.8 | 1.8 |
| hydrochloride | | | | |
| Sulfobutylether beta- | 8.1 | 17.1 | 18.0 | 20 |
| cyclodextrin* | | | | |
| Dextrose anhydrous | 45.5 | 41.8 | 41.4 | 45.5 |
| USP | | | | |
| Citric acid anhydrous, | 0.36 | 0.36 | 0.36 | 0.36 |
| USP | | | | |
| Sodium citrate | 0.18 | 0.18 | 0.18 | 0.18 |
| dihydrate USP | | | | |
| Water for injection | QS | QS | QS | QS |
| Final batch volume (L) | 1500 | 75 | 1800 | 1500 |

*Captisol ®

For each formulation, a clean stainless steel mix tank with overhead stirring was filled to approximately to 20% of final batch volume with Water for Injection (“WFI”). The temperature of the water was adjusted to the set temperature, then citric acid, Captisol®, and sodium citrate dihydrate were added to the mix tank in that order, and mixed for no less than (“NLT”) 5 minutes after each component was added to the mix tank. Amiodarone HCl was added to the mix tank over 10 minutes and mixed for 1 hour using an in-line recirculating high-shear mixer after completing the addition of amiodarone HCl to the mix tank. The tank was brought to 80% of final batch volume with WFI and dextrose was added to the mix tank and mixed for NLT 5 minutes. The tank was brought to 90% of final batch volume with WFI, mixed for NLT 5 minutes and the pH measured. The tank was brought to the final batch volume with WFI, mixed for NLT 5 minutes and the pH measured. The solution was sterile filtered and filled into 200 mL Galaxy® plastic containers and the 20 mg/mL Captisol-containing formulation was also filled into glass bottles. Filled containers were evaluated for the presence of visible particulates.

No gel formation was observed and foaming was substantially reduced and dissipated more quickly as compared to formulations containing 8.1 g/L Captisol. The results of the pH assay and visual inspection are given in the table below.

| | Cyclo- | | | | |
|---|---|---|---|---|---|
| | dextrin | | Cyclo- | | Product |
| | concen- | Temper- | dextrin: | pH at | related |
| | tration | ature | amiodarone | final | particulates |
| Batch | (g/L) | (° C.) | mole ratio | volume | present |
| 10a | 8.1 | 15 | 1.4:1 | 3.59 | Yes |
| 10b | 17.1 | 20-30 | 3:1 | 3.60 | No |
| 10c | 18.0 | 23-27 | 3.2:1 | 3.60 | No |
| 10d | 20.0 | 23 | 3.5:1 | 3.60 | No |

### Example 11

### Formulation Stability During Storage

Formulations 9f and 10c as filled into galaxy bags were stored at 25 and 40° C. and periodically evaluated for presence of product related visible particulates, amiodarone assay and total impurities. The samples stored at 25° C. were evaluated after three and six months storage and the samples stored at 40° C. were evaluated monthly for three months then again at six months. No product related visible particulates were observed. Assay results are included in the table below. Amiodarone was chemically stable with no loss of amiodarone due to adsorption to the container or by degradation to an impurity.

| | Storage | | | | | | |
|---|---|---|---|---|---|---|---|
| | Temperature | | Months | | | | |
| Batch | (° C.) | Test | 0 | 1 | 2 | 3 | 6 |
| 9f | 25 | Assay (% of initial) | 100 | — | — | 99.3 | 100.7 |
| | 25 | Total Impurities (% w/w) | <0.05 | — | — | <0.05 | <0.05 |
| | 40 | Assay (% of initial) | 100 | 100.7 | 100.7 | 100.7 | 102.7 |
| | 40 | Total Impurities (% w/w) | <0.05 | <0.05 | <0.05 | <0.05 | <0.05 |
| 10c | 25 | Assay (% of initial) | 100 | — | — | 99.4 | 100.5 |
| | 25 | Total Impurities (% w/w) | 0.05 | — | — | 0.05 | 0.05 |
| | 40 | Assay (% of initial) | 100 | 100.5 | 100 | 99.4 | 101.1 |
| | 40 | Total Impurities (% w/w) | 0.05 | 0.05 | 0.05 | 0.05 | 0.05 |

### Example 12

### 2-Tank Process

Formulations were prepared using a 2-tank process according to the following formula and procedure:

| | Content in Final Formulation (g/L) | | | | | |
|---|---|---|---|---|---|---|
| | Batch | Batch | Batch | Batch | Batch | Batch |
| Component | 12a | 12b | 12c | 12d | 12e | 12f |
| Amiodarone | 1.8 | 1.8 | 1.8 | 1.8 | 1.8 | 1.8 |
| hydrochloride | | | | | | |
| Sulfobutylether β- | 8.1 | 8.1 | 8.1 | 7.29 | 8.1 | 8.1 |
| cyclodextrin* | | | | | | |
| Dextrose anhydrous | 45.5 | 45.5 | 45.5 | 45.5 | 45.5 | 45.5 |
| USP | | | | | | |
| Citric acid anhydrous, | 0.13 | 0.3 | 0.36 | 0.36 | 0.54 | 0.71 |
| USP | | | | | | |
| Sodium citrate | 0.65 | 0.13 | 0.18 | 0.18 | 0.28 | 0.38 |
| dihydrate USP | | | | | | |
| Water for injection | QS | QS | QS | QS | QS | QS |

*Captisol ®

For each batch, a clean stainless steel vessel (mix tank #1) with overhead stirring was filled with approximately 630 g Water for Injection (“WFI”) and heated to 57° C.±3° C. Captisol® and amiodarone, were added to the mix tank in that order, and mixed until dissolved (8-13 minutes). The solution was observed for presence of gel and none was observed. The tank was cooled to room temperature.

A second clean stainless steel vessel (master tank) with overhead stirring was filled with or 17.5 kg WFI at room temperature. Dextrose, citric acid, and sodium citrate were added, in that order, and dissolved to give a clear colorless solution. The cooled solution from the mix tank #1 was added to the master tank and mixed for 10 minutes. The pH of the solution was measured and adjusted to 3.4-3.6 with 1N hydrochloric acid if needed. The tank was brought to final weight of 25 kg with WFI and mixed for 5 minutes. No gel was observed in the formulation.

Batches 12b-e were filtered through two 0.2 μm Nylon filters in series and filled into 200 mL Galaxy® bags.

### Example 13

### 2-Tank Process with Altered Tank Compositions

Formulations were prepared using a 2-tank process according to the following formula and procedure:

Content (g/L)

| Component | Batch 13a | Batch 13b |
|---|---|---|
| Amiodarone hydrochloride | 1.8 | 1.8 |
| Sulfobutylether beta-cyclodextrin* | 8.1 | 8.1 |
| Dextrose anhydrous USP | 45.5 | 45.5 |
| Citric acid anhydrous, USP | 0.71 | 0.12 |
| Sodium citrate dihydrate USP | 0.38 | 0.076 |
| Water for injection | QS | QS |

*Captisol ®

For each batch, a clean stainless steel vessel (mix tank #1) with overhead stirring was filled with approximately 630 g Water for Injection (“WFI”) and heated to 57° C.±3° C. Citric acid, sodium citrate dihydrate, and Captisol®, were added to the mix tank, and mixed for 5 minutes after each component was added to the mix tank. Amiodarone HCl was added to the mix tank over 2 minutes and mixed for 20 minutes using an overhead stirrer. The tank was cooled to room temperature then the pH adjusted to 3.4-3.6 with 1N hydrochloric acid. The tank was brought to 3 kg with WFI and mixed for 5 minutes. The solution was observed for the formation of gel and none was noted.

A second clean stainless steel vessel (master tank) with overhead stirring was filled with 20 kg (batch 12a) or 17.5 kg (batch 12b) WFI at room temperature. Dextrose was added to the master tank and mixed for 5 minutes. The solution from the mix tank #1 was added to the master tank and mixed for 10 minutes. Mix Tank #1 was twice rinsed with WFI and the rinse solutions were transferred to the master tank. WFI was added to bring the solution weight to approximately 22.5 kg and the solution was mixed for 5 minutes. The pH of the solution was measured and adjusted to 3.4-3.6 with 1N hydrochloric acid. The tank was brought to final weight of 25 kg with WFI and mixed for 5 minutes.

The batches were filtered through two 0.2 μm all Nylon filters in series and filled into 200 mL Galaxy® bags.

### Example 14

### Solubilization of Amiodarone HCl with Different Mole Ratios of a Hydroxyalkyl Ether Cyclodextrin

Formulations were prepared in deionized water according to the following formula:

| | Content (g/L) | | |
|---|---|---|---|
| | Batches | Batch | Batch |
| Component | 14 a-k | 14 m | 14 n |
| Amiodarone hydrochloride | 1.8 | 1.8 | 1.8 |
| 2-hydroxypropyl 4 beta- | 10-40 | 40 | 40 |
| cyclodextrin (“HPCD”) | | | |
| Dextrose (anhydrous) | 45.5 | 45.5 | 45.5 |
| Citric acid (monohydrate) | 0.36 | | |
| Sodium citrate (dihydrate) | 0.18 | | |
| Acetic acid | 9 | 0.64 | |
| Sodium acetate (trihydrate) | | 0.29 | |
| Phosphoric acid (concentrated) | | | 169 μL/L |
| Sodium hydroxide | | | to pH 3-8 |
| Deionized water | QS to 1 L | QS to 1 L | QS to 1 L |

*Captisol ®

An initial 200 mL volume of deionized water was added to the vessels of a Varian VanKel dissolution apparatus and brought to a set temperature. The buffering agents (all 2.5 mM) and HPCD were each added and dissolved with mixing provided by overhead mixing at 250 rpm. Dextrose was also added at this time for batches 14e and 14f. The amiodarone HCl was slowly added with continuing mixing. The solutions were observed for the presence of gel formation and dissolution of the amiodarone and no gel was observed.

Complete dissolution of amiodarone was observed in batches 14c, d and f. When dissolution was complete, the dextrose was added to these batches and they were brought to final volume. The solutions remained clear.

After 6 hours of mixing, the amiodarone in batches 14g, h, and i, was not completely dissolved. These batches were brought to 80% of final volume, the dextrose added and dissolved, and the solutions observed for a further 18 hours. Amiodarone dissolution was incomplete.

Dissolution of the amiodarone in batches 14k, m, and n was incomplete at 6 hours. At 24 hours, dissolution was complete in lots 14m and n so the dextrose was added to these batches and they were brought to final volume. The solutions remained clear.

Formulation parameters and results are indicated in the table below.

| | | | | | Time for |
|---|---|---|---|---|---|
| | HPCD Content | | | Amiodarone | amiodarone |
| | in Final Volume | Buffer | Temp. | dissolution | dissolu- |
| Batch | (g/L) | Type | (° C.) | complete | tion (h) |
| 14a | 10 | Citrate | 30 | No | >24 |
| 14b | 20 | Citrate | 30 | No | >8 |
| 14c | 30 | Citrate | 30 | Yes | 6.5 |
| 14d | 40 | Citrate | 30 | Yes | 3 |
| 14e | 20 | Citrate | 30 | No | >8 |
| 14f | 30 | Citrate | 30 | Yes | 6.5 |
| 14g | 10 | Citrate | 45 | No | >24 |
| 14h | 15 | Citrate | 45 | No | >24 |
| 14i | 20 | Citrate | 45 | No | >24 |
| 14k | 30 | Citrate | 25 | No | >24 |
| 14m | 40 | Acetate | 25 | Yes | 8-24 |
| 14n | 40 | Phos- | 25 | Yes | 8-24 |
| | | phate | | | |

Increasing the HPCD concentration during dissolution by increasing the HPCD:Amiodarone mole ratio decreased the time required for dissolution at a set temperature. An HPCD:Amiodarone mole ratio of about 5.5:1 is required for solubilization of the amiodarone under these conditions.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except by the appended claims. All of the embodiments disclosed and claimed herein can be made and executed without undue experimentation in light of the present disclosure.

What is claimed is:

1. A ready to use injectable intravenous formulation comprising:
an aqueous solution comprising a sulfobutylether beta-cyclodextrin and amiodarone or a pharmaceutically acceptable salt thereof, wherein the sulfobutylether beta-cyclodextrin:amiodarone mole ratio is greater than 2.7:1 and less than or equal to 7:1 and the amiodarone concentration ranges from about 0.7 mM to about 7 mM.

2. The ready to use injectable intravenous formulation of claim 1, wherein the solution is sterile.

3. The ready to use injectable intravenous formulation of claim 1 contained in a bag comprised of polyethylene, polyvinyl chloride, polypropylene, nylon, polyvinylidine chloride or combinations thereof.

4. The ready to use injectable intravenous formulation of claim 1, further comprising one or more components selected from the group consisting of pH adjusting agents, buffering agents, antioxidants, tonicity modifying agents and combinations thereof.

5. The ready to use injectable intravenous formulation of claim 1, wherein the aqueous solution contains about 0.9 mM to about 5 mM citrate buffering agent.

6. The ready to use injectable intravenous formulation of claim 1, wherein the sulfobutylether beta-cyclodextrin:amiodarone mole ratio ranges from about 2.9:1 to about 5:1.

7. The ready to use injectable intravenous formulation of claim 1, wherein the sulfobutylether beta-cyclodextrin concentration ranges from about 13 mg/mL to about 40 mg/mL, and the amiodarone concentration ranges from about 2.2 mM to about 2.7 mM.

8. The ready to use injectable intravenous formulation of claim 1, wherein the surface tension of the aqueous solution ranges from about 59 dynes/cm to about 75 dynes/cm.

9. The ready to use injectable intravenous formulation of claim 1, wherein the aqueous solution has a pH of about 3.6, and comprises about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

10. The ready to use injectable intravenous formulation of claim 1, wherein the aqueous solution has a pH of about 3.6 and comprises about 3 mM citrate buffer, about 2.6 mM amiodarone, about 8.3 mM sulfobutylether-7-beta-cyclodextrin and sufficient dextrose to provide a final solution osmolality of about 255 mOsm/kg to about 345 mOsm/kg.

11. The ready to use injectable intravenous formulation of claim 1, wherein the aqueous solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

12. The ready to use injectable intravenous formulation of claim 1, wherein the aqueous solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

13. The ready to use injectable intravenous formulation of claim 1, wherein there is formation of no more than about 0.1% (w/w) total impurities in the solution when stored at room temperature for 6 months.

14. The ready to use injectable intravenous formulation of claim 1, wherein there is no more than about 1% absorptive loss of amiodarone from the solution when stored at room temperature for 6 months.

15. A method for making a ready to use injectable pharmaceutical composition, the method comprising:
a. preparing an aqueous solution containing a sulfobutylether beta-cyclodextrin and amiodarone or a pharmaceutically acceptable salt thereof, wherein:
i. the sulfobutylether beta-cyclodextrin:amiodarone mole ratio is greater than 2.7:1 and less than or equal to 7:1, and
ii. the amiodarone is dissolved while maintaining a solution temperature of about 15° C. to about 65° C. and a pH less than or equal to the pKa of amiodarone to give a clear solution absent of gel,
b. adding water as needed to provide a final solution with an amiodarone concentration ranging from about 0.7 to 7 mM;
c. sterile filtering the final solution to substantially reduce microbial contamination; and
d. aseptically filling the final solution into a pharmaceutically acceptable container.

16. The method of claim 15, further comprising adding one or more components selected from the group consisting of pH adjusting agents, buffering agents, antioxidants, tonicity modifying agents and combinations thereof to the aqueous solution.

17. The method of claim 15, wherein the final solution contains about 0.9 mM to about 5 mM citrate buffering agent.

18. The method of claim 15, wherein the sulfobutylether beta-cyclodextrin:amiodarone mole ratio ranges from about 2.9:1 to about 5:1.

19. The method of claim 15, wherein the pH during amiodarone dissolution ranges from about 3 to about 4.5.

20. The method of claim 15, wherein the sulfobutylether beta-cyclodextrin concentration in the final solution ranges from about 13 mg/mL to about 40 mg/mL and the amiodarone concentration in the final solution ranges from about 2.2 mM to 2.7 mM.

21. The method of claim 15, wherein the surface tension of the final solution ranges from about 59 dynes/cm to about 75 dynes/cm.

22. The method of claim 15, wherein the final solution has a pH ranging from about 3 to about 4.

23. The method of claim 15, wherein the final solution has a pH of about 3.6, and contains about 3 mM citrate buffer, about 2.2 mM amiodarone, about 6.9 mM sulfobutylether-7-beta-cyclodextrin and sufficient dextrose to provide a final solution osmolality of about 255 mOsm/kg to about 345 mOsm/kg.

24. The method of claim 15, wherein the final solution has a pH of about 3.6 and comprises about 3 mM citrate buffer, about 2.6 mM amiodarone, about 8.3 mM sulfobutylether 7 beta-cyclodextrin and sufficient dextrose to provide a final solution osmolality of about 255 mOsm/kg to about 345 mOsm/kg.

25. The method of claim 15, wherein the final solution has a pH of about 3.6 and comprises about 3 mM citrate buffer, about 2.2 mM amiodarone, about 6.9 mM sulfobutylether-7-beta-cyclodextrin and sufficient sodium chloride to provide a final solution osmolality of about 255 mOsm/kg to about 345 mOsm/kg.

26. The method of claim 15, wherein the final solution has a pH of about 3.6 and comprises about 3 mM citrate buffer, about 2.6 mM amiodarone, about 8.3 mM sulfobutylether-7-beta-cyclodextrin and sufficient sodium chloride to provide a final solution osmolality of about 255 mOsm/kg to about 345 mOsm/kg.

27. The method of claim 15, wherein there is no more than about 1% absorptive loss of amiodarone from the final solution when stored at room temperature for 6 months.

28. The method of claim 15, wherein there is formation of no more than about 0.1% (w/w) total impurities in the final solution when stored at room temperature for 6 months.

29. A method for making a ready to use injectable pharmaceutical composition free of visible particulates, the method comprising:
a. preparing an aqueous solution comprising a sulfobutylether-beta-cyclodextrin and amiodarone or a pharmaceutically acceptable salt thereof, wherein:
i. the sulfobutylether beta-cyclodextrin:amiodarone mole ratio ranges from about 1.1:1 to less than or equal to about 7:1,
ii. the amiodarone is dissolved while maintaining a solution temperature of about 15° C. to less than about 40° C. and a pH less than or equal to the pKa of amiodarone to give a clear solution absent of gel,
b. adding additional sulfobutylether beta-cyclodextrin as needed to provide a sulfobutylether beta-cyclodextrin:amiodarone mole ratio of greater than 2.7:1 to about 7:1; and
c. adding additional water as needed to provide a final solution with an amiodarone concentration ranging from about 0.73 mM to about 7.3 mM.

30. The method of claim 29, wherein the final solution is sterile filtered to substantially reduce the microbial contamination and the filtered solution is aseptically filled into a pharmaceutically acceptable container.

31. The method of claim 29, further comprising adding dextrose to provide a final solution osmolality ranging from about 255 mOsm/kg to about 345 mOsm/kg.

32. The method of claim 29, further comprising adding sodium chloride to provide a final solution osmolality ranging from about 255 mOsm/kg to about 345 mOsm/kg.

33. The method of claim 29, wherein there is formation of no more than about 0.1% (w/w) total impurities in the final solution when stored at room temperature for 6 months.

34. The method of claim 29, wherein there is no more than about 1% absorptive loss of amiodarone from the final solution when stored at room temperature for 6 months.

35. A method for making a ready to use injectable pharmaceutical composition free of visible particulates, the method comprising
a. preparing an aqueous solution containing a sulfobutylether beta-cyclodextrin and amiodarone or a pharmaceutically acceptable salt thereof, wherein:
i. the sulfobutylether beta-cyclodextrin:amiodarone mole ratio ranges from about 1.5:1 to about 7:1,
ii. the amiodarone is dissolved while maintaining a solution temperature ranging from about 15° C. to about 65° C. and a pH less than or equal to the pKa of amiodarone to give a clear solution absent of gel,
b. adding additional sulfobutylether beta-cyclodextrin as necessary to provide a sulfobutylether beta-cyclodextrin:amiodarone mole ratio of greater than about 2.7:1 to about 7:1 in a final solution, and
c. adding additional water as needed to provide the final solution with an amiodarone concentration ranging from 0.73 mM to 7.3 mM.

36. A method for making a ready to use injectable pharmaceutical composition free of visible particulates, the method comprising:
a. preparing a concentrated aqueous solution containing sulfobutylether beta-cyclodextrin and amiodarone or a pharmaceutically acceptable salt thereof, wherein:
i. the volume of the concentrated aqueous solution represents from about 1% to less than about 17% of the volume of a final solution,
ii. the sulfobutylether beta-cyclodextrin:amiodarone mole ratio is ranges from about 1.1:1 to about 7:1,
iii. the amiodarone is dissolved while maintaining a solution temperature of about 15° C. to about 65° C. and a pH less than or equal to the pKa of amiodarone to give a clear solution absent of gel,
b. adding additional sulfobutylether beta-cyclodextrin as necessary to provide the final solution with a sulfobutylether beta-cyclodextrin:amiodarone mole ratio of greater than about 2.7:1 to about 7:1, and
c. adding additional water, as necessary, to provide the final solution with an amiodarone concentration ranging from 0.73 to 7.3 mM.

37. A method for making a ready to use injectable pharmaceutical composition free of visible particulates, the method comprising:
a. preparing a first aqueous solution containing sulfobutylether beta-cyclodextrin, amiodarone or a pharmaceutically acceptable salt thereof, and optionally containing one or more components selected from the group consisting of pH adjusting agents, buffering agents, tonicity modifying agents antioxidants and combinations thereof, wherein:
i. the volume of the first aqueous solution represents from about 1% to less than about 17% of the volume of a final solution,
ii. the sulfobutylether beta-cyclodextrin:amiodarone mole ratio ranges from about 1.1:1 to about 7:1,
iii. the amiodarone is dissolved while maintaining a solution temperature of about 15° C. to about 65° C. and a pH less than or equal to the pKa of amiodarone to give a clear solution absent of gel,
b. preparing a second aqueous solution containing one or more components selected from the group consisting of pH adjusting agents, buffering agents, tonicity modifying agents, antioxidants, additional sulfobutylether beta-cyclodextrin and combinations thereof,
c. combining the two solutions to provide a final solution absent of gel, and
d. adding additional water and sulfobutylether beta-cyclodextrin as necessary to provide a final solution with an amiodarone concentration ranging from 0.73 mM to 7.3 mM and a sulfobutylether beta-cyclodextrin:amiodarone mole ratio of greater than about 2.7:1 to about 7:1.

38. A method for making a ready to use pharmaceutical composition free of visible particulates, the method comprising:
a. adding a volume of water for injection equivalent to 20% to 30% of a volume of a final solution to a suitable container and adjusting the temperature to 20-30° C.,
b. adding and dissolving sulfobutylether 7 beta-cyclodextrin, citric acid and sodium citrate such that the solution pH is in the range of about 3 to about 4,
c. adding and dissolving amiodarone hydrochloride in an amount sufficient to provide a sulfobutylether 7 beta-cyclodextrin:amiodarone mole ratio in the final solution ranging from about 3.1:1 to 3.2:1 to give a clear solution absent of gel,
d. adding additional water for injection to bring the volume of the water to 70-80% of the volume of the final solution, and dissolving sufficient dextrose to provide the final solution with an osmolality ranging from about 255 mOsm/kg to about 345 mOsm/kg,
e. adjusting the pH to 3.55-3.65 with hydrochloric acid or sodium hydroxide,
f. adding additional water for injection to bring the volume of the water to the volume of the final solution wherein the amiodarone concentration ranges from 2 mM to 3 mM and the citrate concentration ranges from about 2 mM to 3 mM,
g. sterile filtering the final solution to substantially reduce microbial contamination, and
h. aseptically filling the final solution into a pharmaceutically acceptable container.

39. The method of claim 38, where the pharmaceutically acceptable container is a flexible bag comprised of one or more of ethylene vinyl acetate, polyolefin, polypropylene, polyethylene, polyvinylchloride, nylon, polyvinylidene chloride, or combinations thereof.

40. The method of claim 38, wherein the final solution comprises amiodarone at a concentration ranging from about 2 mM to about 2.4 mM and the citrate concentration ranges from about 2 mM to about 3 mM.

41. The method of claim 38, wherein the final solution comprises amiodarone at a concentration ranging from about 2.5 mM to about 2.8 mM and the citrate concentration ranges from about 2 mM to about 3 mM.
